(19) United States
(12) Patent Application Publication
(10) Pub. No.: US 2008/0058245 A1
(43) Pub. Date: Mar. 6, 2008

(54) Vectors for Recombinant Protein Expression in E. Coli

(76) Inventors: Karl F. Johnson, Hatboro, PA (US); Dan Bezila, Quakertown, PA (US); Winnie Ngo, Hatboro, PA (US); David Hakes, Willow Grove, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/585,440

(22) PCT Filed: Jan. 6, 2005
(86) PCT No.: PCT/US05/00302

§ 371 Date: May 8, 2007

Related U.S. Application Data
(60) Provisional application No. 60/535,263, filed on Jan. 9, 2004.

(51) Int. Cl. C12P 21/06; A61K 38/02; C12N 15/70

(52) U.S. Cl. 514/2; 435/320.1; 435/69.1

(57) ABSTRACT

The present invention relates to methods of providing a protein product to a customer. In particular, the invention relates methods of using protein expression vectors to produce proteins to be provided to a client. The invention also provides vectors for the cloning and expression of proteins, including reagent proteins and therapeutic proteins.

### CROSS-REFERENCES TO RELATED APPLICATIONS

This application is a U.S. National Phase Application, filed under 35 U.S.C. §371 of Patent Cooperation Treaty Application Number PCT/US2005/00302, filed Jan. 6, 2005, and claims priority to U.S. Provisional Application No. 60/535,263, filed Jan. 9, 2004. Each of the aforementioned applications are hereby incorporated by reference in their entirety and for all purposes.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

Humans have exploited the use of genetics and microorganisms for their own advantage throughout much of recorded history. Egyptians are credited with the first use of yeast to produce leavened bread sometime between 4000-2000 BC, Gregor Mendel produced peas having specific, defined, characteristics in the mid-19th century, and the Food and Drug Administration approved the first recombinant drug, human insulin, in 1982. This last feat is often considered to be the beginning of the modern biotechnology industry. Since then, transgenic plants, recombinant foods, recombinant vaccines, cancer therapeutics, recombinant antibodies, enzymes, glycosyltransferases, cytokines, coagulation factors, hormones, dermal replacements, anti-virals, and many other recombinant proteins have been developed for human use.

The nucleic acid expression vector has greatly aided in the production of recombinant proteins and therapeutics. A nucleic acid encoding a protein reagent or therapeutic protein can be cloned into an expression vector, which can be expressed in a population of eukaryotic and/or prokaryotic cells, thus producing a large amount of a recombinant protein or therapeutic. However, the yield and quality of the recombinant product depend greatly on the expression vector and microorganism used to express the vector. In addition, the use of recombinant cells can be slow and tax resources that can be otherwise used for discovery and improvement of recombinant proteins and therapeutics.

As the demand and usefulness of recombinant proteins increases, new methods are required in order to more efficiently prepare such proteins with a rapid turnaround time. Moreover, as recombinant proteins for the treatment of a variety of diseases are generated, methods to lower the cost of their production need to be implemented so that these technologies are available to all those in need. The need to provide improved vectors for protein expression does not exist solely in the therapeutic protein arena. Rather, this need also extends to the production of proteins or reagents(e.g., enzymes) for use in the production of both protein and non-protein therapeutics.

Over the past several decades, recombinant proteins and therapeutics have proven to be the answer in treating many diseases that were not addressed using conventional, chemical therapeutics. However, recombinant technology has been hampered by inefficiency, especially in small scale situations, as well as high cost and slow turnaround time. Streamlining the expression of proteins at a lower cost with a quicker turnaround time for virtually any customer situation is needed to realize the potential of recombinant proteins as reagents and as therapeutics. The present invention meets this need.

### BRIEF SUMMARY OF THE INVENTION

The invention includes a method of providing a therapeutic protein to a customer, comprising cloning a nucleic acid encoding a protein into a pCWin1 expression vector as set forth in SEQ ID NO:1, expressing a protein therefrom, and providing the protein to a customer.

In another aspect of the invention, a method of providing a therapeutic protein to a customer comprises cloning a nucleic acid encoding a protein into a pCWin2 expression vector as set forth in SEQ ID NO:2, expressing a protein therefrom, and providing the protein to a customer.

In yet another aspect of the invention, a method of providing a therapeutic protein to a customer comprises cloning a nucleic acid encoding a protein into a pCWin2/MBP expression vector as set forth in SEQ ID NO:3, expressing a protein therefrom, and providing the protein to a customer. In still another aspect, a method of providing a therapeutic protein to a customer comprises cloning a nucleic acid encoding a protein into a pCWin2-MBP-SBD (PMS₃₉) expression vector as set forth in SEQ ID NO:10, expressing a protein therefrom, and providing the protein to a customer. In yet another aspect of the invention, a method of providing a therapeutic protein to a customer comprises cloning a nucleic acid encoding a protein into a pCWin2-MBP-MCS-SBD (PMXS₃₉) expression vector as set forth in SEQ ID NO:11, expressing a protein therefrom, and providing the protein to a customer.

In an embodiment of the invention, a pCWIN2/MBP vector comprises a protease cleavage site coding sequence between the MBP coding sequence and the therapeutic protein coding sequence.

Therapeutic proteins useful in the present invention include erythropoietin, human growth hormone, granulocyte colony stimulating factor, interferons alpha, -beta, and -gamma, Factor IX, follicle stimulating hormone, interleukin-2, erythropoietin, anti-TNF-alpha, and lysosomal hydrolases such as beta-glucosidase, alpha-galactosidase-A, beta-hexosaminidase, beta- gal acto sidase, alpha-gal acto sidase, alpha-mannosidase, beta-mannosidase, alpha-L- fucosidase, beta-glucuronidase, alpha-glucosidase, alpha-N-acetylgalactosaminidase, and acid phosphatase.

In one embodiment of the invention, a method of providing a protein to a customer includes cloning a nucleic acid encoding a protein into a pCWin1 expression vector as set forth in SEQ ID NO:1, expressing a protein therefrom, and providing the protein to a customer.

In another embodiment of the invention, a method of providing a protein to a customer includes cloning a nucleic acid encoding a protein into a pCWin2 expression vector as set forth in SEQ ID NO:2, expressing a protein therefrom, and providing the protein to a customer.

In another embodiment of the invention, a method of providing a protein to a customer includes cloning a nucleic acid encoding a protein into a pCWin2/MBP expression vector as set forth in SEQ ID NO:3, expressing a protein therefrom, and providing the protein to a customer. In still another aspect, a method of providing a protein to a customer comprises cloning a nucleic acid encoding a protein into a pCWin2-MBP-SBD (PMS₃₉) expression vector as set forth in SEQ ID NO:10, expressing a protein therefrom, and providing the protein to a customer. In yet another aspect of the invention, a method of providing a protein to a customer comprises cloning a nucleic acid encoding a protein into a pCWin2-MBP-MCS-SBD (PMXS₃₉) expression vector as set forth in SEQ ID NO:11, expressing a protein therefrom, and providing the protein to a customer.

In one aspect of the invention, a protein may be a glycosyltransferase or a sugar nucleotide-generating enzyme.

In an aspect of the invention, an expression vector includes an affinity tag coding sequence. In this aspect of the invention, an affinity tag may be a histidine tag, a Factor IX tag, a glutathione-S-transferase tag, starch-binding domain and a FLAG-tag.

The invention includes a method of providing a therapeutic protein to a customer, where the method includes providing an expression vector to a protein production facility wherein a nucleic acid encoding a protein is cloned into the expression vector and the protein is expressed therefrom in the protein production facility; subsequently providing the protein to a customer. In an aspect of the invention, the expression vector comprises a multiple-cloning region and an antibiotic resistance marker. The antibiotic resistance marker may be kanamycin, tetracycline, or chloramphenicol. In another aspect of the invention, the expression vector includes an affinity tag.

In an embodiment of the invention, a method of providing a protein to a customer includes providing a pCWin1 vector as set forth in SEQ ID NO:1 to a protein production facility, wherein a nucleic acid encoding a protein is cloned into the expression vector and the protein is expressed therefrom in the protein production facility, and the protein is provided to a customer.

In an embodiment of the invention, a method of providing a protein to a customer includes providing a pCWin2 vector as set forth in SEQ ID NO:2 to a protein production facility, wherein a nucleic acid encoding a protein is cloned into the expression vector and the protein is expressed therefrom in the protein production facility, and the protein is provided to a customer.

In an embodiment of the invention, a method of providing a protein to a customer includes providing a pCWin2/MBP vector as set forth in SEQ ID NO:3 to a protein production facility, wherein a nucleic acid encoding a protein is cloned into the expression vector and the protein is expressed therefrom in the protein production facility, and the protein is provided to a customer. In another embodiment of the invention, a method of providing a protein to a customer includes providing a pCWin2-MBP-SBD (pMS₃₉) vector as set forth in SEQ ID NO:10 to a protein production facility, wherein a nucleic acid encoding a protein is cloned into the expression vector and the protein is expressed therefrom in the protein production facility, and the protein is provided to a customer. In still another embodiment of the invention, a method of providing a protein to a customer includes providing a pCWin2-MBP-MCS-SBD (PMXS₃₉) vector as set forth in SEQ ID NO:11 to a protein production facility, wherein a nucleic acid encoding a protein is cloned into the expression vector and the protein is expressed therefrom in the protein production facility, and the protein is provided to a customer.

In one aspect of the invention, a protein production facility is in-house. In another aspect of the invention, the protein production facility is offsite.

The invention also includes a method of providing a protein to a customer, wherein at least one glycosyl moiety is added to a protein prior to providing the protein to a customer.

In one aspect, a glycosyl moiety is added to a protein in vitro.

The present invention includes a method of providing a protein to a customer, comprising cloning a nucleic acid encoding said protein into a pCWin1 expression vector as set forth in SEQ ID NO:1, inserting the vector into a bacterial host cell, expressing the protein in the host cell, and providing the protein to a customer.

Another embodiment of the invention includes a method of providing a protein to a customer, comprising cloning a nucleic acid encoding said protein into a pCWin2 expression vector as set forth in SEQ ID NO:2, inserting the vector into a bacterial host cell, expressing the protein in the host cell, and providing the protein to a customer.

Another embodiment of the invention includes a method of providing a protein to a customer, comprising cloning a nucleic acid encoding said protein into a pCWin2/MBP expression vector as set forth in SEQ ID NO:3, inserting the vector into a bacterial host cell, expressing the protein in the host cell, and providing the protein to a customer. Yet another embodiment of the invention includes a method of providing a protein to a customer, comprising cloning a nucleic acid encoding said protein into a pCWin2-MBP-SBD (pMS₃₉) expression vector as set forth in SEQ ID NO:10, inserting the vector into a bacterial host cell, expressing the protein in the host cell, and providing the protein to a customer. Still another embodiment of the invention includes a method of providing a protein to a customer, comprising cloning a nucleic acid encoding said protein into a pCWin2-MBP-MCS-SBD (PMXS₃₉) expression vector as set forth in SEQ ID NO:11, inserting the vector into a bacterial host cell, expressing the protein in the host cell, and providing the protein to a customer.

In one embodiment of the invention, a method includes adding at least one glycosyl moiety to a protein prior to providing the protein to a customer. In one aspect, a glycosyl moiety is added to a protein in vitro.

The invention features isolated pcWINI expression vector comprising the sequence set forth in SEQ ID NO:1. The invention also features an isolated pcWINl expression vector consisting of the sequence set forth in SEQ ID NO:1.

In another aspect, the invention features an expression vector comprising the sequence set forth in SEQ ID NO:2. The invention also features an isolated pcWIN2 expression vector consisting of the sequence set forth in SEQ ID NO:2.

In yet another aspect, the invention features an isolated pcWIN2/MBP expression vector comprising the sequence set forth in SEQ ID NO:3. The invention also features an isolated pcWIN2/MBP expression vector consisting of the sequence set forth in SEQ ID NO:3. The invention further features a pcWIN2/MBP expression vector, wherein the pCWIN2/MBP vector comprises a protease cleavage site coding sequence adjacent to the MBP coding sequence.

In another aspect, the invention features an isolated pCWin2-MBP-SBD (pMS₃₉) expression vector comprising the sequence set forth in SEQ ID NO:10. The invention also features an isolated pCWin2-MBP-SBD (pMS₃₉) expression vector consisting of the sequence set forth in SEQ ID NO:10.

In still another aspect, the invention features an isolated pCWin2-MBP-MCS-SBD (pMXS₃₉) expression vector comprising the sequence set forth in SEQ ID NO:11. The invention also features an isolated pCWin2-MBP-MCS-SBD (PMXS₃₉) expression vector consisting of the sequence set forth in SEQ ID NO:11.

The invention features a method of expressing a protein from an isolated pcWIN1 expression vector comprising the sequence set forth in SEQ ID NO:1. In another embodiment, the invention features a method of expressing a protein from an isolated pcWIN2 expression vector comprising the sequence set forth in SEQ ID NO:2. In yet another embodiment, the invention features a method of expressing a protein from an isolated pcWIN2/MBP expression vector comprising the sequence set forth in SEQ ID NO:3. In still another embodiment, the invention features a method of expressing a protein from an isolated pCWin2-MBP-SBD (PMS₃₉) expression vector comprising the sequence set forth in SEQ ID NO:10. In another embodiment, the invention features a method of expressing a protein from an isolated pCWin2-MBP-MCS-SBD (PMXS₃₉) expression vector comprising the sequence set forth in SEQ ID NO:11. In one aspect, the protein is expressed in a prokaryotic cell.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiment(s) which are presently preferred. It should be understood, however, that invention is not limited to the precise arrangements and instrumentalities shown. In the drawings:

FIG. 1A is an image of an electrophoretic gel containing products of a restriction digest. Lanes 1 and 3 are BstEII DNA Marker, lane 2 is SacI/XbaI-digested Cst-04 vector and lane 4 is Kan^{r} PCR product digested with SacI/XbaI.

FIG. 1B is an image of an agar plate showing the result of Cst-04-Kan^{r} transformation plated on LB kan^{r} plate.

FIG. 1C is an image of an electrophoretic gel containing DNA from an E. coli colony that screened positive for the Cst-04-Kan^{r} insert. Lane 1 contains BstEII DNA Marker. Lanes 2-4 contain DNA isolated from the Cst-04-Kan5 colony. Lane 2 contains DNA cut with NdeI, lane 3 contains DNA cut with SalI, and lane 4 contains DNA cut with Pst1.

FIG. 1D is an image of an ampicillin-containing agar plate and a kanamycin-containing agar plate, on both of which Cst-04-Kan5 was streaked. The ampicillin-containing plate inhibited the growth of Cst-04-Kan5-containing cells, demonstrating that the ampicillin gene in Cst-04-Kan5-containing cells is inactive, whereas the kanamycin-containing plate supports the growth of Cst-04-Kan5-containing cells, demonstrating that the kanamycin gene in Cst-04-Kan5-containing cells is operative.

FIG. 1E is an image of thin-layer chromatography of the products of the activity of Cst-04Kan5 plasmid-containing cell lysates using lacto-N-neotetraose as a substrate. Lanes labeled 1 and 2 are Cst-04Kan5 from JM109 cells, lanes labeled 3 are Cst-04Kan5 isolated from TGI cells, and lanes labeled 4 are Cst-04-6-1.

FIG. 2A is an image of the agarose gel from which restriction enzyme-digested PCR products were isolated. Lanes marked “M” contain 1 kb DNA markers, lane 1 contained pCWINI insert, lane 2 contained NdeI/ScaI-digested pCWori Kan^{r} Cst04Kan5 vector, lane 3 contained pre-pCWIN2 insert, and lane 4 contained BamHI/EcoRI-digested pCWori Kan^{r} Cst04Kan5 vector.

FIG. 2B is an image of an electrophoretic gel, illustrating the results of restriction digestion of plasmid DNA isolated from positive transformants as a result of pCWin1 and pre-pCWin2 DNA mini-prep. Lanes labeled “M” contain 1 kb DNA markers. Lanes 1 to 5 contain pCWin1 clones. Lanes 6 to 14 contains pre-pCWin2 clones. All 14 clones were digested with EcoR1.

FIG. 2C is an image of an electrophoretic gel, illustrating the results of restriction digestion of plasmid DNA isolated from positive transformants as a result of pCWin1 and pre-pCWin2 DNA mini-prep. Lanes labeled “M” contain 1 kb DNA markers. Lane 1 contains pCWin1 clone #5, lane 2 contains pre-pCWin2 clone #11. Both clones #5 and #11 were digested with NdeI and ScaI.

FIG. 2D is an image of an electrophoretic gel, illustrating the results of restriction pCWin2 mini-prep screening. Lanes labeled “M” contains 1 kb DNA markers. Lanes 1 through 18 contain pCWin2 clones. The clones were all digested with PstI.

FIG. 3A is an image of an electrophoretic gel containing the NdeI/BamHI-digested ma1E cDNA.

FIG. 3B is an image of an electrophoretic gel containing the restriction enzyme-digested pCWin2 vector.

FIG. 3C is an image of two electrophoretic gels containing the restrction enzyme-digested pCWin2 vector. This figure represents the screening of colonies to verify that the ma1E NdeI and BamHI insert size was correct. The first lane on each gel contains 1 kb DNA molecular weight markers, as indicated in the figure. Lanes 1, 2, 3, 4, 5, 7, 8, 9 and 10 correspond to colonies selected from the transformation plate and which positively show the presence of the ma1E cDNA. Lane 6 corresponds to a colony selected from the transformation plate bearing a vector that does not contain the ma1E insert.

FIGS. 4A, 4B, and 4C comprise the entire nucleotide sequence of pcWIN1, as set forth in SEQ ID NO:1.

FIGS. 5A, SB, and SC comprise the entire nucleotide sequence of pcWIN2, as set forth in SEQ ID NO:2.

FIGS. 6A, 6B, 6C, and 6D comprise the entire nucleotide sequence of pcWIN2/MBP, as set forth in SEQ ID NO:3.

FIG. 7A is an image of an electrophoretic gel illustrating the results of a restriction enzyme-digested PCR reaction used to create the SBD₃₉ insert. Lane M is a 1 kb DNA marker. Lane 1 is the SBD₃₉ PCR insert product digested with Bgl II and BamH1. The expected size for the SBD₃₉ insert is 447 bp.

FIG. 7B is an image of an electrophoretic gel illustrating the result of the restriction enzyme digestion of pCWin2-MBP-ST3Gal III (A73) vector. Lane M is a 1 kb DNA marker. Lane 1 is pCWin2-MBP-ST3Gal III (A73) digested with BamHI. The expected size for the vector is 7 kb.

FIG. 7C is an image of an electrophoretic gel illustrating the results of the DNA mini-prep enzymatic digestion screen of pCWin2-MBP-SBD-ST3Gal III (A73). Lanes M are a 1 kb DNA marker, lanes 1 through 11 are pCWin2MBP-SBD-ST3Gal III A73 construct colonies 1 through 11 respectively, digested with Ndel and BamHl. The expected size for the pCWin2-ST3Gal III A73 vector band is 5.9 kb and the expected size for the MBP-SBD insert is 1.6 kb. Clone #6 (Lane 6) illustrates a positive result.

FIG. 7D is an image of an electrophoretic gel illustrating the restriction enzyme digestion of pCWin2 vector with BamHI and ScaI. Lane M is a 1 kb DNA marker. Lane 1 is digested pCWin2. The expected size for the vector is 4.3 kb and the expected size for the BamHI/ScaI MCS insert is 0.8 kb.

FIG. 7E is an image of an electrophoretic gel illustrating the results of the restriction enzyme digestion of pCWin-MBP-SBD₃₉-ST3Gal III (A73). Lane M is a 1 kb DNA marker. Lane 1 is pCWin-MBP-SBD₃₉-ST3Gal III (A73) digested with BamHI and ScaI.

The expected size for the vector is Linear is pCWin-MBP-SBD₃₉ is 5.8 kb and the expected size for the BamHI /ScaI ST3Gal III (A73) insert is 1.6 kb.

FIG. 7F is an image of an electrophoretic gel illustrating the results of the DNA mini-prep restriction enzyme digestion screen of pCWin2-MBP-SBD₃₉ clones. Lanes M is a 1 kb DNA marker, lanes 1 through 16 are pCWin2-MBP-SBD₃₉ construct colonies 1 through 16 respectively, digested with Nde I and Xba I. The expected size for the pCWin2 vector band is 5.0 kb and the expected size for the MBP-SBD insert is 1.65 kb. Clone #1 (Lane 1) illustrates a positive result.

FIG. 7G is a nucleic acid vector feature map, illustrating restriction sites for the pCWin-MBP-SBD₃₉ (PMS₃₉) construct.

FIG. 8A is an electrophoretic gel illustrating the results of the PCR reaction to prepare the SBD insert. Lane M is X BstE II DNA marker, lane 1 is SBD PCR insert product. The expected size for the SBD insert is 447 bp.

FIG. 8B is an image of an electrophoretic gel illustrating the results of DNA isloated from PCR-Blunt-SBD colonies and subjected to restriction enzyme digestion. Lane M is a X BstE II DNA marker, lanes 1 through 8 are PCR-Blunt-SBD colonies 13, 14, 15, 17, 18, 19, 20, and 22 respectively, all digested with Xhol and Sall. The expected size for the PCR-Blunt vector band is 3 kb and the expected size for the SBD insert is 447 bp.

FIG. 8C is an image of an electrophoretic gel illustrating the results of the restriction enzyme-digested pCWin2-MBP kan^{r} vector. Lane M is a lkb DNA ladder, lane 1 is pCWin2-MBP kan^{r} Vector digested with XhoI and Sal I. The expected size for the pCWin2-MBP kan^{r} vector band is 3 kb.

FIG. 8D is an image of an electrophoretic gel illustrating the results of DNA isolated from pCWin2-MBP-MCS-SBD (pMXS₃₉) vector-containing colonies. Lane M is a), BstE II DNA marker, lanes 1 through 13 are pMXS39 colonies 1 through 13 respectively, digested with Xho I and Sall. The expected size for the pCWin2-MBP vector band is 6.1 kb and the expected size for the SBD insert is 447 bp. Two out of thirteen colonies had the correct size of insert and pMXS₃₉ vector (see lanes 4 and 5 in FIG. 8D).

FIG. 8E is a nucleic acid vector feature map, illustrating restriction sites for the pCWin-MBP-MCS-SBD₃₉ (PMXS₃₉) construct.

### DETAILED DESCRIPTION OF THE INVENTION

The use of therapeutic proteins to treat patients experiencing disease or illness increases yearly. Protein therapeutics typically lack the same problematic side effects found with certain traditional chemical therapeutics. Even in instances where the protein therapeutic is altered slightly from its natural state, such a protein typically does not have the same side effects as do certain chemical therapeutics. Similar to the increase in the use of therapeutic proteins, the use of non-therapeutic, or “reagent” proteins increases exponentially from year to year. For example, reagent proteins are used in such areas as food biochemistry, bioremediation, production of small molecule therapeutics, and even in the production of therapeutic proteins.

The increasing use of protein reagents and therapeutics has enhanced the need for production and preparation of such proteins. It is generally impractical, in terms of cost and time, to isolate and purify a protein therapeutic from its natural source. The cost of isolating proteins from natural sources is prohibitive, and the amount of time needed for such isolation techniques is lengthy. For example, a difficult and time-consuming process for isolating a therapeutic protein from a natural source will drive up the cost of that reagent or therapeutic, which in the latter instance, may unduly burden a medical patient, the patient's insurer, or both. Further, a burdensome isolation process can limit the amount of therapeutic protein available to those in need thereof. Finally, a difficult isolation process can also overburden the entity that produces the reagent or therapeutic protein, reducing profits and wasting valuable business time.

In vitro systems have therefore been developed to produce recombinant forms of reagent proteins and therapeutic proteins. One of the most significant groups of organisms used as an in vitro system for production of recombinant therapeutic proteins is bacteria, and in particular, Escherichia coli. E. coli is often used for its simplicity, as it is easy to culture and to maintain, and more importantly, it is easy to manipulate genetically. Further, it is relatively simple to isolate protein expressed from E. coli.

There are numerous expression vectors that are compatible with bacteria, and in particular, with E. coli, for the purpose of producing recombinant therapeutic proteins. However, many vectors are useful only under particular circumstances, and therefore have drawbacks with respect to their utility for selected protein expression under the specific circumstances that may be required. The present invention sets forth methods of providing a protein to a customer that overcome some of the difficulties associated with commercial protein production.

The present invention therefore features a method of providing a protein to a customer, wherein the protein of interest is expressed in a vector containing a nucleic acid encoding the protein of interest, as well as a multiple cloning site and an antibiotic resistance marker, and further wherein the resulting protein is provided to a customer. Part of the advantage of the present invention is that the expression vectors of the present invention reduce the complexity of subcloning the cDNA encoding a therapeutic protein. Further, the expression vector of the present invention enables the production of proteins using an antibiotic resistance marker other than the ampicillin antibiotic resistance marker, which is not approved for Good Manufacturing Practice (GMP) protocols required by the Food and Drug Administration.

Part of the advantage of the present invention is due to the flexibility of the expression vector used to express the protein. The flexibility of a vector of the present invention provides that a protein can be produced rapidly and efficiently. A cDNA encoding a protein of interest can be readily subcloned into the expression vector by way of a multiple cloning site. Therefore, a method of the present invention features the use of an expression vector as described above to provide a protein to a customer in a more efficient manner.

Another advantage of the present invention is that expression vectors of the invention offer increased productivity and efficiency of protein expression. That is, the design and use of vectors of the present invention provide increased levels of protein expression and production, leading to increased efficiencies in protein expression over similar vectors known in the art. Such advantages increase the quantity of protein produced, and therefore also serve to lower the cost of protein production and increase profit through sales of protein.

The flexibility and functionality of a vector of the present invention increases the ease, efficiency and reliability of the delivery of a protein to a customer. The use of a method of the present invention to streamline and enhance protein product delivery to a customer not only increases the production and profitability of a business entity using such methods, but it also has the effect of increasing the opportunity for medical patients in need thereof with a therapeutic protein.

The present invention also features vectors for expression of reagent proteins, and methods of providing reagent proteins produced using such vectors to a customer. Vectors of the invention useful for the expression of reagent proteins may be the same vectors used to produce therapeutic proteins in methods of the invention. Additionally, vectors of the invention useful for the expression of reagent proteins may be different vectors than those used to produce therapeutic proteins in methods of the invention.

Vectors of the invention designed for production of reagent proteins are further useful for production of proteins that are themselves useful in the subsequent production of small chemical therapeutics and for remodeling of non-protein molecules, such as carbohydrates. Examples of such proteins include glycosyltransferases, glycosidases and enzymes used in the production of sugar nucleotides. Further, such proteins are useful to produce and remodel carbohydrate-containing glycoproteins. The production and remodeling of glycoproteins has significant therapeutic impact, as glycoproteins form the basis of a significant number of recombinant therapeutics.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

An “affinity tag” is a peptide or polypeptide that may be genetically or chemically fused to a second polypeptide for the purposes of purification, isolation, targeting, trafficking, or identification of the second polypeptide. The “genetic” attachment of an affinity tag to a second protein may be effected by cloning a nucleic acid encoding the affinity tag adjacent to a nucleic acid encoding a second protein in a nucleic acid vector.

As used herein, the term “glycosyltransferase,” refers to any enzyme/protein that has the ability to transfer a donor sugar to an acceptor moiety.

A “sugar nucleotide-generating enzyme” is an enzyme that has the ability to produce a sugar nucleotide. Sugar nucleotides are known in the art, and include, but are not limited to, such moieties as UDP-Gal, UDP-GlcNAc, and CMP-NAN.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′ end; the left-hand direction of a double-stranded polynucleotide sequence is referred to as the 5′-direction.

A first defined nucleic acid sequence is said to be “immediately adjacent to” a second defined nucleic acid sequence when, for example, the last nucleotide of the first nucleic acid sequence is chemically bonded to the first nucleotide of the second nucleic acid sequence through a phosphodiester bond. Conversely, a first defined nucleic acid sequence is also said to be “immediately adjacent to” a second defined nucleic acid sequence when, for example, the first nucleotide of the first nucleic acid sequence is chemically bonded to the last nucleotide of the second nucleic acid sequence through a phosphodiester bond.

A first defined polypeptide sequence is said to be “immediately adjacent to” a second defined polypeptide sequence when, for example, the last amino acid of the first polypeptide sequence is chemically bonded to the first amino acid of the second polypeptide sequence through a peptide bond. Conversely, a first defined polypeptide sequence is said to be “immediately adjacent to” a second defined polypeptide sequence when, for example, the first amino acid of the first polypeptide sequence is chemically bonded to the last amino acid of the second polypeptide sequence through a peptide bond.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, “homology” is used synonymously with “identity.” The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul et al. (1990, J. Mol. Biol. 215:403-410), and can be accessed, for example, at the BLAST site of the National Center for Biotechnology Information (NCBI) world wide web site at the National Library of Medicine (NLM) at the National Institutes of Health (NIH). BLAST nucleotide searches can be performed with the NBLAST program (designated “blastn” at the NCBI web site), using the following parameters: gap penalty=5; gap extension penalty=2; mismatch penalty=3; match reward=1; expectation value10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used as available on the website of the National Center for Biotechnology Information of the National Library of Medicine at the National Institutes of Health.

The percent identity between two sequences can be determined using techniques similar to those described above, with or without allowing gaps. In calculating percent identity, typically exact matches are counted.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “therapeutic protein” as the term is used herein refers to any protein that is useful to treat a disease state or to improve the overall health of a living organism. A therapeutic protein may effect such changes in a living organism when administered alone, or when used to improve the therapeutic capacity of another substance.

A “reagent protein” as the term is used herein refers to any protein that is useful in food biochemistry, bioremediation, production of small molecule therapeutics, and even in the production of therapeutic proteins. Typically, reagent proteins are enzymes capable of catalyzing a reaction to produce a product useful in any of the aforementioned areas.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

A “multiple cloning site” as the term is used herein is a region of a nucleic acid vector that contains more than one sequence of nucleotides that is recognized by at least one restriction enzyme.

An “antibiotic resistance marker” as the term is used herein refers to a sequence of nucleotides that encodes a protein which, when expressed in a living cell, confers to that cell the ability to live and grow in the presence of an antibiotic.

The term “saccharide” refers in general to any carbohydrate, a chemical entity with the most basic structure of (CH₂O)ₙ. Saccharides vary in complexity, and may also include nucleic acid, amino acid, or virtually any other chemical moiety existing in biological systems.

“Monosaccharide” refers to a single unit of carbohydrate of a defined identity.

“Oligosaccharide” refers to a molecule consisting of several units of carbohydrates of defined identity. Typically, saccharide sequences between 2-20 units may be referred to as oligosaccharides.

“Polysaccharide” refers to a molecule consisting of many units of carbohydrates of defined identity. However, any saccharide of two or more units may correctly be considered a polysaccharide.

A “party” as the term is used herein refers to an individual or an entity involved in a transaction related to a method, a vector, or a protein of the present invention. For example, an individual who provides a vector to a business entity is considered to be a “party” in the context of the present invention. Further, the business entity is also considered to be a “party” in the context of the present invention.

A “recipient” as the term is used herein refers to a specific. party who receives a vector or a protein of the present invention. For example, if an individual gives a business entity a vector of the invention, the business entity is considered to be a “recipient” in the context of the present invention. By way of another example, an individual within an organization may provide a second individual within the same organization with a vector or a protein of the present invention. The second individual, who is in receipt of a vector or a protein of the invention, is considered to be a “recipient” in terms of the present invention. It should be noted that a recipient may be a customer, but that not all customers are recipients.

A “customer,” as the term is used herein, refers to an intended recipient of a specific item in a formal transaction. A customer is also a recipient, but is distinct from a recipient in that a customer is recipient who is an endpoint for a transaction, whereas a recipient may be an intermediate in a larger transaction. For example, customers of the present invention include, but are not limited to, an entity responsible for the creation of an expression vector that contains the cDNA encoding a protein of interest, if that entity will use the protein produced. A customer is also an entity responsible for expression of a protein from a vector that contains the cDNA encoding a protein of interest, if that entity will use the protein produced. A customer also may be an entity that purchases a protein expressed from a vector of the present invention, for the purpose of using the protein. Further, the entity that creates an expression vector of the present invention may be a customer if that entity uses the protein produced by the vector.

A “protein production facility” as the term is used herein is any location that has the ability to express a protein encoded within a nucleic acid vector.

As the term is used herein, “in-house” refers to dealings within a single organization. In this context, an organization may be a single company, two or more jointly cooperating laboratories, or a corporation and its subsidiaries, collectively.

“Offsite,” as the term is used herein, refers to dealings that extend beyond the “in-house” context.discussed above. For example, the transfer of a vector of the invention from a first organization to a second organization is a transfer of that vector “offsite.”

### I. Vectors

The present invention includes an isolated nucleic acid encoding a protein operably linked to a nucleic acid comprising a promoter/regulatory sequence such that the nucleic acid is capable of directing expression of the protein encoded by the nucleic acid. Thus, the invention encompasses expression vectors and methods for the expression of proteins based on exogenous DNA introduced into cells with concomitant expression of the exogenous DNA in the cells such as those described, for example, in Sambrook et al. (1989, supra), and Ausubel et al. (1997, supra).

An expression vector of the present invention is based on the pcWori+ vector (Muchmore et al., 1987, Meth. Enzymol. 177:44-73). However, the pcWori+ vector by itself is not adequate for the production of protein reagents and therapeutic proteins according to the present invention. The pcWori+ vector contains an ampicillin resistance marker. Certain regulatory agencies require that the production of proteins for therapeutic use cannot be carried out using recombinant vectors containing ampicillin resistance genes. Therefore, an expression vector of the present invention features an antibiotic resistance marker approved by the U.S. Food and Drug Administration (FDA) for use in the production of protein reagents and therapeutic proteins. Such- antibiotic resistance markers include, but are not limited to, kanamycin, tetracycline, and chloramphenicol.

In the invention, the ampicillin resistance marker normally present in the pcWori+ vector is disabled as follows. Briefly, the ampicillin resistance marker in the pcwori+ vector is disrupted in order to produce, in part, a vector of the present invention. PCR primers designed to create Pvu1 and Sca1 restriction enzyme cleavage sites on either end of a kanamycin resistance gene are used, and the resultant PCR product is digested with Pvu1 and Scal restriction enzymes. The ampicillin resistance gene in a pcWori+ vector is also cut with Pvul and Scal restriction enzymes. Subsequently, the kanamycin resistance gene is ligated into the pcWori+ vector that was cleaved within the ampicillin resistance gene.

Verification of successful disruption of the ampicillin resistance gene and successful insertion of the kanamycin resistance gene is observed by transforming E. coli cells, for example, with the ligation mixture. Growth of the transformed cells on kanamycin-containing agar plates confirms the successful insertion of the kanamycin resistance marker, while lack of growth on ampicillin containing plates confirms the successful disruption of the ampicillin resistance gene. Other methods of disruption or deletion of the ampicillin resistance gene will be known to one of skill in the art. Similarly, other methods of inserting the kanamycin resistance gene, or any other antibiotic resistance gene useful in the present invention, and methods of confirming the insertion and/or deletion of genes will also be known to one of skill in the art.

Another feature of a vector of the present invention is a versatile and highly-functional multiple cloning site. As described in, for example, in Sambrook et al. (1989, supra), a “multiple cloning site” is a nucleic acid having a sequence encoding more than one restriction enzyme recognition site. The practical purpose of a multiple cloning site is to allow the ligation (i.e., “insertion”) of an exogenous polynucleotide into the multiple cloning site, wherein the exogenous polynucleotide may have different restriction enzyme recognition sequences at its 5′ and 3′ ends. That is, the multiple cloning site allows flexibility with respect to the identity of the 5′ and 3′ ends on an exogenous polynucleotide, thus facilitating the cloning of such a polynucleotide into the multiple cloning site.

A multiple cloning site is most often found, and is most useful, in a nucleic acid vector such as a vector of the present invention. As will be known to the skilled artisan, a multiple cloning site may be located adjacent to other functional elements in a vector, such as a promoter. A multiple cloning site may also be designed such that insertion of an exogenous polynucleotide into the multiple cloning site results in the exogenous polynucleotide being expressed in frame with the adjacent elements to create a fusion protein of the protein encoded by the exogenous polynucleotide and the protein encoded by the adjacent element.

Accordingly, a vector of the present invention contains at least one multiple cloning site. The creation of a functional multiple cloning site in a vector of the present invention is described in greater detail elsewhere herein. Briefly, a multiple cloning site may be designed and synthesized de novo, or it may be isolated from another pre-existing vector. PCR methods are used to create multiple cloning site polynucleotides having specific restriction enzyme recognition sites on either end (5′ and 3′) of the multiple cloning site polynucleotide. A multiple cloning site polynucleotide is then inserted into a pcWori+ vector of the present invention by means of specific restriction enzyme recognition sites corresponding to those on either end of the multiple cloning site polynucleotide. As will be understood by one skilled in the art, various molecular biological techniques are available to insert, delete, and/or modify a multiple cloning site in a vector of the present invention in order to create a more functional and flexible multiple cloning site useful in connection with the present invention.

Another feature of a vector of the present invention is the option of an affinity tag coding sequence located in the multiple cloning site. An affinity tag coding sequence may be inserted into the multiple cloning site adjacent to, upstream from, or downstream from a target protein coding sequence. As will be understood by one of skill in the art, an affinity tag will typically be inserted into the multiple cloning site in frame with the target protein.

One of skill in the art will also understand that an affinity tag coding sequence can be used to produce a recombinant fusion protein by concomitantly expressing the affinity tag and target protein. The expressed fusion protein can then be isolated, purified, or identified by means of the affinity tag. An affinity tag is especially important when expressing proteins that are reagents and less important when expressing therapeutic proteins due to restrictions imposed by regulatory agencies.

Affinity tags useful in the present invention include, but are not limited to, a maltose binding protein, a histidine tag, a Factor IX tag, a glutathione-S-transferase tag, a FLAG-tag, and a starch binding domain tag. Other tags are well known in the art, and the use of such tags in the present invention would be readily understood by the skilled artisan.

Any single vector of the present invention may have more than one feature described herein. By way of a non-limiting example, a vector of the present invention may have a disrupted ampicillin resistance gene, a functional kanamycin resistance gene, and a modified, multi-functional multiple cloning site. An example of one such vector of the present invention is pCWin1, the sequence of which is set forth in SEQ ID NO:1. A pCWin1 vector of the present invention has, for example, two BamHI restriction enzyme recognition sites, one of which is located within the multiple cloning site. Another example of a vector of the present invention is pCWin2, the sequence of which is set forth in SEQ ID NO:2. A pCWin2 vector of the present invention has, for example, only one BamHI restriction enzyme recognition site which is located within the multiple cloning site.

A further example of a vector of the present invention is pCWin2-MBP, the sequence of which is set forth in SEQ ID NO:3. A pCWin2-MBP vector of the invention has, for example, one BamHI restriction enzyme recognition site located within the multiple cloning site and additionally has an E. coli ma1E maltose binding protein coding sequence inserted into the multiple cloning site in between the NdeI and BamHI restriction enzyme recognition sites. The NdeI sequence in the multiple cloning site contains an ATG start codon. The pCWin2-MBP vector is therefore useful, for example, for expression of a fusion protein comprised of a maltose binding protein and a desired protein. This is achieved by inserting a polynucleotide encoding the desired protein into the multiple cloning site in frame with the maltose binding protein and expressing the entire open reading frame encoded in the multiple cloning site.

Yet another example of a vector of the present invention is pCWin2-MBP-SBD₃₉ (PMS₃₉), the sequence of which is set forth in SEQ ID NO:10. A pCWin2-MBP-SBD₃₉ (pMS₃₉) vector of the invention has, for example, one BamHI restriction enzyme recognition site located within the multiple cloning site, and one EcoRI restriction enzyme recognition site located within the multiple cloning site, and additionally has an E. coli ma1E maltose binding protein coding sequence inserted into the multiple cloning site in between the NdeI and Sacd restriction enzyme recognition sites. The pCWin2-MBP-SBD₃₉ (PMS₃₉) vector also has a starch-binding domain (SBD) inserted between the EcoRI and BamHI restriction sites.

The NdeI sequence in the multiple cloning site contains an ATG start codon. The pCWin2-MBP-SBD₃₉ (pMS₃₉) vector is therefore useful, for example, for expression of a fusion protein comprised of a maltose binding protein, a starch binding domain, and a desired protein. This is achieved by inserting a polynucleotide encoding the desired protein into the multiple cloning site in frame with the maltose binding protein and starch binding domain, and expressing the entire open reading frame encoded in the multiple cloning site.

Still another example of a vector of the present invention is pCWin2-MBP-MCS-SBD₃₉ (pMXS₃₉), the sequence of which is set forth in SEQ ID NO:11. As compared to, the pMXS₃₉ vector expresses, in one aspect of the invention, a fusion protein with the structure “MBP-desired protein-SBD,” whereas the pMS₃₉ vector expresses, in another aspect of the invention, a “MBP-SBD-esired protein” fusion protein. Accordingly, a pCWin2-MBP-SBD₃₉ (pMXS₃₉) vector of the invention has, for example, one XhoI restriction enzyme recognition site located within the multiple cloning site, and one SalI restriction enzyme recognition site located within the multiple cloning site, and additionally has an E. coli ma1E maltose binding protein coding sequence inserted into the multiple cloning site in between the NdeI and Sacd restriction enzyme recognition sites. The pCWin2-MBP-SBD₃₉ (PMXS₃₉) vector also has a starch-binding domain (SBD) inserted between the XhoI and SalI restriction sites. The NdeI sequence in the multiple cloning site contains an ATG start codon. The pCWin2-MBP-SBD₃₉ (PMXS₃₉) vector is therefore useful, for example, for expression of a fusion protein comprised of a maltose binding protein, a starch binding domain, and a desired protein. This is achieved by inserting a polynucleotide encoding the desired protein into the multiple cloning site in frame with the maltose binding protein and starch binding domain, and expressing the entire open reading frame encoded in the multiple cloning site.

A vector of the present invention, as described above, is useful for the production of a therapeutic protein. A polynucleotide sequence encoding a therapeutic protein may be inserted into the multiple cloning site using any technique known to the skilled artisan. For example, a polynucleotide sequence encoding a therapeutic protein may be modified to contain specific restriction enzyme recognition sites at the 5′ and 3′ ends of the polynucleotide. Such restriction enzyme recognition sites will correspond to recognition sites located within the multiple cloning site of a vector of the present invention, facilitating the insertion (by ligation) of the therapeutic protein-encoding sequence into the multiple cloning site, and when expressed, producing a therapeutic protein.

Therapeutic proteins useful in the present invention are numerous and are well-known in the art, and are therefore not listed here. By way of a non-limiting example, such therapeutic proteins include erythropoietin, human growth hormone, granulocyte colony stimulating factor, interferons alpha, -beta, and -gamma, Factor IX, follicle stimulating hormone, interleukin-2, erythropoietin, anti-TNF-alpha, and a lysosomal hydrolase. Lysosomal hydrolases useful in the present invention include, but are not limited to, beta-glucosidase, alpha-galactosidase-A, beta-hexosaminidase, beta-galactosidase, alpha-galactosidase, alpha-mannosidase, beta-mannosidase, alpha-L-fucosidase, beta-glucuronidase, alpha-glucosidase, alpha-N-acetylgalactosaminidase, and acid phosphatase. It will be understood that any mutant or variant of a therapeutic protein may be expressed using vectors of the present invention.

The present invention also features a vector useful for the production of a non-therapeutic protein, referred to herein as reagent proteins. As will be understood by the skilled artisan, a reagent protein is one which does not currently have a therapeutic application. Such proteins include, but are not limited to, enzyme reagents, food enzymes, nutritional supplements, and non-active additives. Methods of expressing reagent proteins using vectors of the invention will be understood by the skilled artisan to be conducted in the same manner as the above-described methods of expressing therapeutic proteins using vectors of the present invention.

Another feature of a vector of the present invention is the option of a protease cleavage site coding sequence located in the multiple cloning site. A protease cleavage site coding sequence may be inserted into the multiple cloning site adjacent to, upstream from, or downstream from a target protein coding sequence. As will be understood by one of skill in the art, a protease cleavage site will typically be inserted into the multiple cloning site in frame with the target protein. One of skill in the art will also understand that a protease cleavage site coding sequence can be used to produce a recombinant fusion protein by concomitantly expressing the protease cleavage site sequence and target protein. The expressed fusion protein can then be isolated, purified, or identified by means of the protease cleavage site.

In an embodiment of the invention, a vector contains a coding sequence for a protease cleavage site which is located C-terminal to the nucleic acid sequence encoding an MBP. In one aspect, a vector is pCWin2-MBP-SBD₃₉ (PMS₃₉). In another aspect, a vector is pCWin2-MBP-MCS-SBD₃₉ (pMXS₃₉).

A fusion protein containing a preselected protease cleavage site, as will be understood by one of skill in the art, is useful for the removal of amino acid sequence that is extraneous or non-essential to the expressed protein of interest. For example, a target protein may be expressed using a vector of the present invention as a fusion with an affinity tag for the purpose of purification of the target protein, but the affinity tag may not be desirable once the protein is sufficiently purified. The insertion of a specific protease cleavage site between the target protein and the affinity tag is useful for the cleavage of the affinity tag from the target protein. Protease cleavage sites useful in the present invention include, but are not limited to, an enterokinase cleavage site, a Factor Xa cleavage site, a thrombin cleavage site, and a TEV protease cleavage site. The skilled artisan will understand the characteristics and uses of a protease cleavage useful in the present invention.

The present invention also features a recombinant bacterial host cell comprising, inter alia, a nucleic acid vector as described elsewhere herein. In one aspect, the recombinant cell is transformed with a vector of the present invention. The transformed vector need not be integrated into the cell genome nor does it need to be expressed in the cell. However, the transformed vector will be capable of being expressed in the cell. In one aspect of the invention, E. coli is used for transformation of a vector of the present invention and expression of protein therefrom. In another aspect of the invention, a K-12 strain of E. coli is useful for expression of protein from a vector of the present invention. Strains of E. coli useful in the present invention include, but are not limited to, JM83, JM101, JM103, JM109, W3110, chi 1776, and JA221.

It will be understood that a host cell useful in the present invention will be capable of growth and culture on a small scale, medium scale, or a large scale. For example, a host cell of the invention is useful for testing the expression of a protein from a vector of the invention equally as much as it is useful for large scale production of a reagent or therapeutic protein product. Techniques useful in culturing host cells and expressing protein from a vector contained therein are well known in the art and will therefore not be listed herein.

A host cell of the present invention may be transformed with a vector of the present invention to produce a transformed host cell of the invention. Transformation, as known to the skilled artisan, includes the process of inserting a nucleic acid vector into a host cell, such that the host cell containing the nucleic acid vector remains viable. Such transformation of nucleic acid into a bacterial cell is useful for purposes including, but not limited to, creation of a stably-transformed host cell, making a biological deposit, propagating the vector-containing host cell, propagating the vector-containing host cell for the production and isolation of additional vector, expression of target protein encoded by vector, and the like.

Methods of transforming a vector are numerous and well-known in the art, and will therefore not be listed here. By way of a non-limiting example, a competent bacterial cell of the invention may be transformed by a vector of the invention using electroporation. Methods of making bacterial cells “competent” are well-known in the art, and typically involve preparation of the bacterial cells so that the cells take up exogenous DNA. Similarly, methods of electroporation are known in the art, and detailed descriptions of such methods may be found, for example, in Sambrook et al. (1989, supra). The transformation of a competent cell with vector DNA may be also accomplished using chemical-based methods. One example of a well-known chemical-based method of bacterial transformation is described by Inoue, et al. (1990, Gene 96:23-28). Other methods of transformation will be known to the skilled artisan.

In one embodiment of the present invention, a Cst-04Kan5 plasmid is transfomed into E. coli JM109 cells using 20 μl JM109 competent cells in 0.34 μl 1.42 M beta mercaptoethanol, incubated on ice for 10 minutes, at which time 1 μl (100 ng) Cst-04-Kan5 plasmid is added to the transformation mixture. The cell/DNA mixture is incubated ice for 30 minutes, then heat shocked at 42° C. for 45 seconds. The reaction is then incubated on ice for 2 minutes, at which time 80 μl SOC media is added. The reaction mixture is then shaken at 37° C. for 1 hour, and subsequently, plated on LB Kan^{r} agar plates. Identification and confirmation of the Cst-04-Kan5 plasmid DNA is carried out using a restriction enzyme digestion of plasmid DNA isolated from positive transformants, using NdeI, SalI, PstI restriction enzymes.

A transformed host cell of the present invention may be used to express a protein. In an embodiment of the invention, a transformed host cell contains a vector of the invention, which contains therein a nucleic acid sequence encoding an exogenous protein. The protein is expressed using any expression method known in the art (for example, IPTG). The expressed protein may be contained within the host cell, or it may be secreted from the host cell into the growth medium.

Methods for isolating an expressed protein are well-known in the art, and the skilled artisan will know how to determine the best method for isolation of an expressed protein based on the characteristics of any given host cell expression system. By way of a non-limiting example, an expressed protein that is secreted from a host cell may be isolated from the growth medium. Isolation of a protein from a growth medium may include removal of bacterial cells and cellular debris. By way of another non-limiting example, an expressed protein that is contained within a host cell may be isolated from the host cell. Isolation of such an “intracellular” expressed protein may include disruption of the host cell and removal of cellular debris from the resultant mixture. These methods are not intended to be exclusive representations of the present invention, but rather, are merely for the purposes of illustration of various applications of the present invention.

Purification of a protein expressed in accordance with the present invention may be effected by any means known in the art. The skilled artisan will know how to determine the best method for the purification of a protein expressed in accordance with the present invention. A purification method will be chosen by the skilled artisan based on factors such as, but not limited to, the expression host, the contents of the crude extract of the protein, the size of the protein, the properties of the protein, the desired end product of the protein purification process, and the subsequent use of the end product of the protein purification process.

In an embodiment of the invention, isolation or purification of a protein expressed in accordance with the present invention may not be desired. In an aspect of the present invention, an expressed protein may be stored or transported inside the bacterial host cell in which the protein was expressed. In another aspect of the invention, an expressed protein may be used in a crude lysate form, which is produced by lysis of a host cell in which the protein was expressed. In yet another embodiment of the invention, an expressed protein may be partially isolated or partially purified according to any of the methods set forth or described herein. The skilled artisan will know when it is not desirable to isolate or purify a protein of the invention, and will be familiar with the techniques available for the use and preparation of such proteins.

### II. Methods of Providing a Protein to a Customer

The present invention features a method of providing a protein to a customer. In an embodiment of the invention, a nucleic acid encoding a protein is cloned into an expression vector. The encoded protein is expressed from the expression vector, and the resulting protein product is provided to a customer.

In an embodiment of the invention, a protein is expressed from an expression vector in vitro. Techniques for in vitro protein expression are known in the art, and are exemplified by the methods of Melton and colleagues (Krieg et al., 1987, Meth. Enzymol. 155, 397-415; Yisraeli et al., 1989, Meth. Enzymol. 180, 42-50). A protein produced using an in vitro expression method of the present invention is provided to a customer.

In another embodiment of the invention, a protein is expressed from an expression vector in vivo. Numerous techniques for expression of a protein from an expression vector in vivo are described in detail elsewhere herein, and are also well-known in the art. Such techniques include, but are not limited to, expression of a protein from a vector in a bacterial host cell. A protein produced using an in vivo expression method of the present invention is provided to a customer.

In one aspect of the invention, a protein is expressed from a pcWIN1 expression vector, as set forth in SEQ ID NO:1. In another aspect of the invention, a protein is expressed from a pcWIN2 expression vector, as set forth in SEQ ID NO:2. In yet another aspect of the invention, a protein is expressed from a pcWIN2/MBP expression vector, as set forth in SEQ ID NO:3. In still another aspect of the invention, a protein is expressed from a pCWin2-MBP-SBD₃₉ (PMS39) expression vector, as set forth in SEQ ID NO:10. In yet another aspect of the invention, a protein is expressed from a pCWin2-MBP-MCS-SBD₃₉ (PMXS₃₉) expression vector, as set forth in SEQ ID NO:11. As will be understood by one of skill in the art, a pcWIN vector of the present invention is useful in any of the expression methods set forth herein for the production of a target protein that may be provided to a customer.

Methods of the present invention for in vivo expression of a protein in a bacterial cell comprise transformation of the bacterial cell with an expression vector comprising the protein of interest. Methods of transforming a bacterial cell with a vector are described in detail elsewhere herein, and would be understood by one of ordinary skill in the art. It will be appreciated that methods of bacterial cell transformation other than those explicitly disclosed herein are useful in methods of the present invention, and therefore, are within the scope of the present invention.

Vectors featured in methods of the present invention are described in detail elsewhere herein. In an embodiment of the invention, a method of providing a protein to a customer comprises expressing a protein from an expression vector useful for production of a therapeutic protein. As described above, vectors of the invention useful in such methods are comprised of an antibiotic resistance marker such as kanamycin, tetracycline, chloramphenicol, and the like, as such antibiotics are particularly useful in connection with the expression of therapeutic proteins. A therapeutic protein provided to a customer using this method is particularly useful to the customer due to the fact that kanamycin, tetracycline, chloramphenicol and like antibiotics are preferred by certain regulatory agencies for the production of therapeutic proteins.

The present invention therefore features a method of providing a known therapeutic protein to a customer. Therapeutic proteins include, but are not limited to, human growth hormone, granulocyte colony stimulating factor, interferons alpha, -beta, and -gamma, Factor IX, follicle stimulating hormone, beta-glucosidase, interleukin-2, erythropoietin, alpha-galactosidase-A, and anti-TNF-alpha. It will be understood by the skilled artisan that any nucleic acid encoding a therapeutic protein, wherein the nucleic acid is capable of being cloned into and expressed from a nucleic acid vector of the invention, will be useful in the present invention. The ability to determine a therapeutic protein useful in the present invention is within the skill of the ordinary artisan and such a determination does not require undue experimentation.

In another embodiment of the invention, a method of providing a protein to a customer comprises expressing a protein from an expression vector useful for production of a reagent protein. Vectors of the invention useful in such methods preferably have had the native ampicillin resistance gene disabled, altered, or deleted, such that the ampicillin resistance gene is no longer functional in the vector. Such vectors are comprised of any antibiotic resistance marker other than ampicillin known in the art to be useful in the expression of proteins. Antibiotic resistance markers useful in the invention include kanamycin, tetracycline, chloramphenicol, and like antibiotic resistance markers approved by certain regulatory agencies, as well as any antibiotic resistance marker not approved by certain regulatory agencies for use with therapeutic proteins.

Therefore, the present invention also features methods of providing a protein to a customer, wherein the protein is a reagent protein, and therefore, need not be expressed from a vector containing antibiotic resistance marker accepted by a regulatory agency. However, a reagent protein may also be expressed from a vector containing an FDA-accepted antibiotic resistance marker. A protein produced by such a method of the invention may be useful for almost any purpose, including, but not limited to, an enzyme reagent, a food enzyme, a nutritional supplement, and a non-active additive. Examples of such proteins include, but are not limited to a glycosyltransferase and a sugar nucleotide-generating enzyme.

In an embodiment of the invention, a method is provided wherein a nucleic acid is cloned into a vector containing any antibiotic resistance marker useful in the expression of a protein, the protein is expressed therefrom, and the resulting protein product is provided to a customer. It will be understood that such a protein may be expressed in vivo or in vitro.

Methods of the present invention also feature a vector comprising a highly-functional multiple cloning site. Such vectors are described in detail elsewhere herein. In an embodiment of the invention, a method of providing a protein to a customer comprises expressing a protein from an expression vector useful for production of a therapeutic protein.

As described above, vectors of the invention useful in such methods are comprised of a highly-functional multiple cloning site, in addition to an antibiotic resistance marker such as kanamycin, tetracycline, chloramphenicol, and the like. In this embodiment, a therapeutic protein is expressed cloned into a vector comprising a highly-functional multiple cloning site in addition to an antibiotic resistance marker, expressed therefrom, and provided to a client. In one aspect of the invention, the multiple cloning site contains at least one of NdeI, BamHI, SacI, HindIII, XbaI, XhoI, EcoRI, KpnI, and SalI restriction enzyme cleavage sites.

Methods of the present invention also feature a vector comprising an affinity tag. Such vectors are described in detail elsewhere herein. In an embodiment of the invention, a method of providing a protein to a customer comprises expressing a protein from an expression vector useful for production of a therapeutic protein. As described above, vectors of the invention useful in such methods are comprised of an affinity tag, in addition to an antibiotic resistance marker such as kanamycin, tetracycline, chloramphenicol, and the like. In this embodiment, a therapeutic protein is expressed cloned into a vector comprising an affinity tag in addition to an antibiotic resistance marker, expressed therefrom, and provided to a client. In a preferred embodiment, the affinity tag is a maltose-binding protein.

In other embodiments of the invention, a useful affinity tag may be, but is not limited to, a histidine tag, a Factor IX tag, a glutathione-S-transferase tag, starch-binding domain, a FLAG-tag, and the like. One of skill in the art will understand that any affinity tag capable of being used with a vector of the present invention will be useful in methods of the invention. Further, the skilled artisan will also appreciate that a single vector of the invention may comprise more than one affinity tag, and that multiple affinity tags may be identical or may be heterogeneous in sequence.

The present invention also features a method of providing a protein to a customer, wherein an expression vector used to express a protein has multiple characteristics as described elsewhere herein. For example, a method of the present invention for providing a protein to a customer comprises the cloning of a nucleic acid encoding the protein into an expression vector, wherein the expression vector comprises a kanamycin resistance marker and a highly-functional multiple cloning site.

In an embodiment of the invention, a method of providing a protein to a customer comprises the cloning of a protein into the expression vector set forth in SEQ ID NO:1. The protein-SEQ ID NO:1 construct is transformed into an E. coli cell, the protein is expressed therefrom, and the protein product is provided to the customer. In one aspect of the invention, the E. coli cell is a JM109 cell. In another aspect of the invention, the protein is a therapeutic protein. In yet another aspect of the protein, the protein is a reagent protein.

In another embodiment of the invention, a method of providing a protein to a customer comprises the cloning of a protein into the expression vector set forth in SEQ ID NO:2. The protein-SEQ ID NO:2 construct is transformed into an E. coli cell, the protein is expressed therefrom, and the protein product is provided to the customer. In one aspect of the invention, the E. coli cell is a JM109 cell. In another aspect of the invention, the protein is a therapeutic protein. In yet another aspect of the protein, the protein is a reagent protein.

In another embodiment of the invention, a method of providing a protein to a customer comprises the cloning of a protein into the expression vector set forth in SEQ ID NO:3. The protein-SEQ ID NO:3 construct is transformed into an E. coli cell, the protein is expressed therefrom, and the protein product is provided to the customer. In one aspect of the invention, the E. coli cell is a JM109 cell. In another aspect of the invention, the protein is a therapeutic protein. In yet another aspect of the protein, the protein is a reagent protein.

In still another embodiment of the invention, a method of providing a protein to a customer comprises the cloning of a protein into the expression vector set forth in SEQ ID NO:10. The protein-SEQ ID NO:10 construct is transformed into an E. coli cell, the protein is expressed therefrom, and the protein product is provided to the customer. In one aspect of the invention, the E. coli cell is a JM109 cell. In another aspect of the invention, the protein is a therapeutic protein. In yet another aspect of the protein, the protein is a reagent protein.

In another embodiment of the invention, a method of providing a protein to a customer comprises the cloning of a protein into the expression vector set forth in SEQ ID NO:11. The protein-SEQ ID NO:11 construct is transformed into an E. coli cell, the protein is expressed therefrom, and the protein product is provided to the customer. In one aspect of the invention, the E. coli cell is a JM109 cell. In another aspect of the invention, the protein is a therapeutic protein. In yet another aspect of the protein, the protein is a reagent protein.

The present invention features a method of providing a protein to a customer, wherein a nucleic acid encoding a protein is cloned into an expression vector of the invention by the party providing a vector to a recipient. That is, a nucleic acid encoding a protein is cloned into an expression vector of the invention before the vector is transferred to a recipient. In an embodiment of the invention, a method of providing a protein to a customer comprises providing a vector to a recipient, wherein the vector contains a nucleic acid encoding a protein. The recipient of the vector expresses the protein, and the protein is then provided to a customer. In one aspect of the invention, a recipient is a protein production facility.

In an embodiment of the invention, a method of providing a protein to a customer comprises providing a vector to a recipient, wherein the vector does not contain a nucleic acid encoding a protein. The recipient of the vector clones a nucleic acid encoding a protein into the vector and expresses the protein, and the protein is then provided to a customer. In one aspect of the invention, a recipient is a protein production facility. In another aspect of the invention, the nucleic acid cloned into a vector is provided by the party providing the vector to the recipient. In yet another aspect of the invention, the nucleic acid cloned into a vector is provided by the recipient.

The invention also features a method of providing a protein to a customer, wherein the method comprises providing a vector to a recipient, wherein the vector comprises a nucleic acid encoding a protein, for the purpose of expression of the protein encoded by the vector. In one embodiment of the invention, the recipient is a protein production facility. In one aspect, the protein production facility is in-house. By way of non-limiting examples, such recipients include an in-house protein production facility and an in-house laboratory. In another aspect of the invention, the protein production facility is offsite. By way of non-limiting examples, such recipients include an offsite protein production facility, an offsite laboratory, an offsite biotechnology company and an offsite pharmaceutical company.

A protein produced using a method of the present invention may be provided to a customer by the recipient of the vector, wherein the recipient is responsible for expressing the protein from the vector provided by another party. Alternatively, a protein produced using a method of the present invention may be provided to a customer by the original party that provided the vector to the recipient, wherein the recipient expresses the protein and provides the resulting protein product to the original party so that the original party may provide the protein to a customer.

A protein produced using a method of the present invention may be provided to a customer in the form of a purified protein, a partially purified protein, an isolated protein, a partially isolated protein, a bacterial cell lysate, cell paste or purified inclusion bodies. It will be understood that a protein produced using a method of the present invention may be provided to a customer in any form known in the art to be useful for the storage, transfer, or processing of a recombinant protein.

The present invention also features a method of providing a protein to a customer, wherein at least one glycosyl moiety is added to the protein before providing the protein to the customer. A glycosyl moiety may be added to a protein using any method known in the art. Additionally, a glycosyl moiety may be added to a protein of the invention using any one of the methods or reagents taught by DeFrees et al. in PCT Application WO 03/031464, which is incorporated herein by reference in its entirety. The skilled artisan will understand, based on the disclosure herein, that any of the methods known in the art or set forth herein are useful for glycosylating a protein of the present invention prior to providing the protein to a customer.

Thus, in an embodiment of the present invention, a method of providing a protein to a customer comprises providing a vector comprising a nucleic acid encoding a protein to a recipient, wherein the recipient expresses the protein, and further wherein the protein is modified with at least one glycosyl moiety before the protein is provided to a customer. In one aspect of the invention, at least one glycosyl moiety is added to the protein by the recipient before providing the protein to a customer. In another aspect of the invention, at least one glycosyl moiety is added to the protein by the recipient before providing the protein to the original supplier of the vector, wherein the original supplier of the vector subsequently provides the protein to a customer. In yet another aspect of the invention, the recipient provides the expressed protein to the original supplier of the vector, wherein the original supplier of the vector adds at least one glycosyl moiety to the protein before providing the protein to a customer.

In another embodiment of the invention, a method of providing a protein to a customer comprises providing a vector to a recipient, wherein the recipient clones a nucleic acid encoding a protein into the vector, expresses the protein, and further wherein the protein is modified with at least one glycosyl moiety before the protein is provided to a customer. In one aspect of the invention, at least one glycosyl moiety is added to the protein by the recipient before providing the protein to a customer. In another aspect of the invention, at least one glycosyl moiety is added to the protein by the recipient before providing the protein to the original supplier of the vector, wherein the original supplier of the vector to the recipient provides the protein to a customer. In yet another aspect of the invention, the recipient provides the expressed protein to the original supplier of the vector, wherein the original supplier of the vector adds at least one glycosyl moiety to the protein before providing the protein to a customer.

### EXPERIMENTAL EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

### Example 1

### Modification of pCWori+Amp^{r} Expression Vector by Disrupting the Amp^{r} Gene and Adding the Kanamycin Resistance Gene

The pCWori+Amp^{r} vector contains an ampicillin resistance marker, as well as the genes encoding N. Meningitidis CMP-NAN synthetase (CNS) and Campylobacter Jejuni α2,3 Sialyl Transferase (CstI), referred to as Cst-04 (Cst-04 was provided by Warren Wakarchuck, National Research Council, Canada). This example describes the complete process by which the Cst-04 (pCWori+amp^{r}-CNS-CstI) plasmid was interrupted at the PvuI and Scal sites of ampicillin gene by the insertion of the kanamycin resistance gene.

A kanamycin resistance gene was isolated from pGEX-Kt-ext Kan^{r} using PCR to generate cDNA with modified restriction sites at 5′ (Pvu1-ATTCCAATTCGATCGGGGGGGGGGGGAAA) (SEQ ID NO:4) and 3′ (ScaI-ATTCCAAGTAGTACTTTAGAAAAACTCATCG) (SEQ ID NO:5) ends. The PCR product was then subcloned into a Cst04 (pCWori+amp^{r}-CNS-CstI) vector in TG1 cells. A colony positive for the recombinant vector (Cst-04Kan5) was identified, and the Cst-04Kan5 plasmid was isolated, then transformed into JM 109 cells.

A PCR reaction was conducted containing 1 ng pGEXKan^{r} template, 1 μg (1 μl) kan-ScaI/Pvu1 primer, 77 μl H₂O, 8 μl dNTP mixture, 10 μl 10× buffer, and 1 μl Vent polymerase. The reaction parameters included a 5 minute cycle at 95° C., followed by the addition of 1 μl of Vent polymerase and thirty cycles of the following temperature pattern: 94° C. for one minute, 55° C. for one minute, 72° C. for one minute.

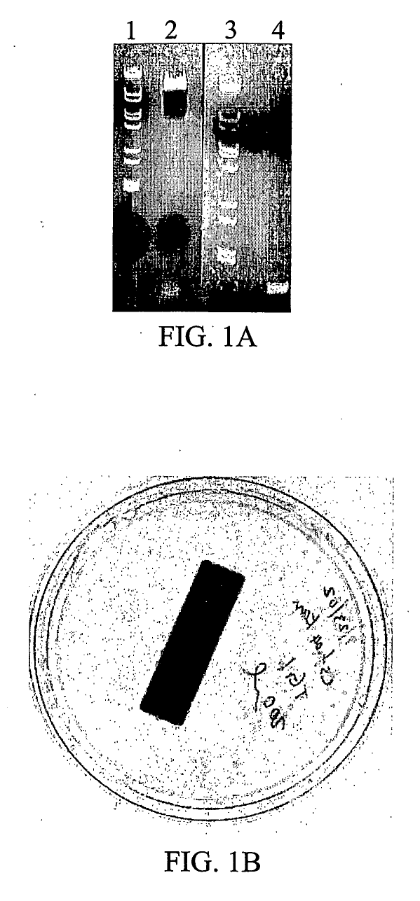

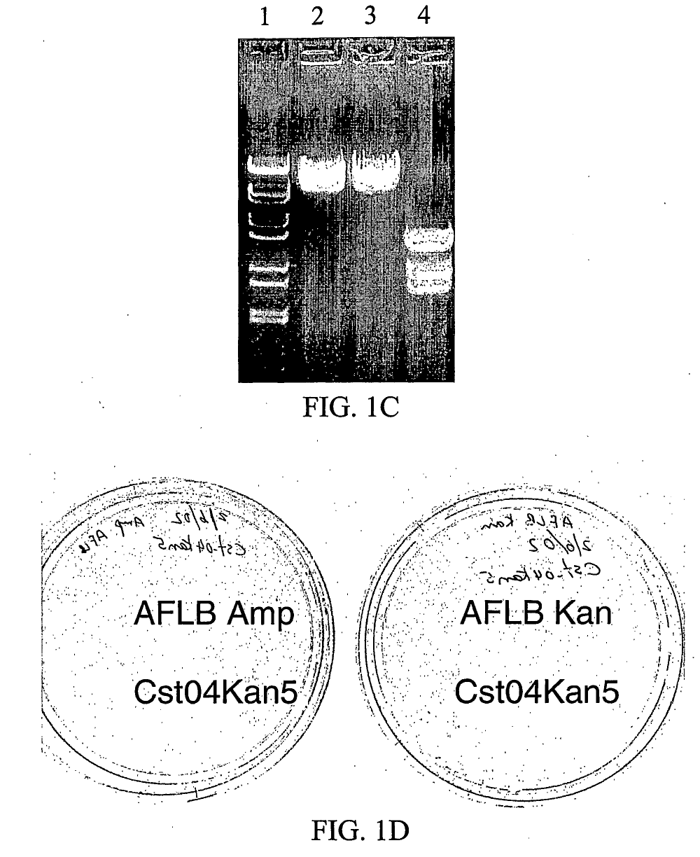

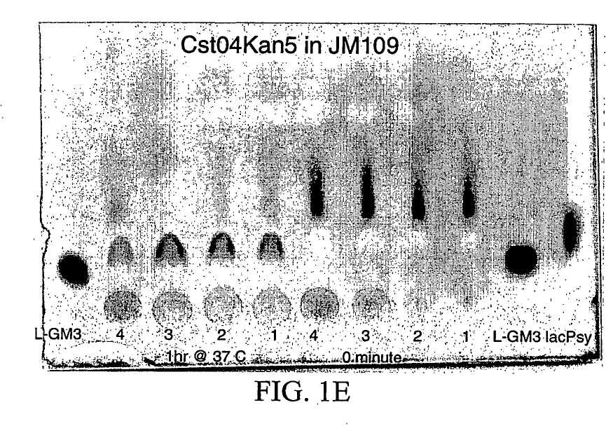

The PCR product and the Cst-04 vector were subjected to a restriction digest. The PCR product digest included 16 μl PCR r×n, 2 μl 10× buffer, 0.5 μl Pvu1, 0.5 μl ScaI, 1 μl H₂O. The pGEX-Ktext Kan^{r} vector digest included 1 μl pGEX-Ktext Kan^{r} vector, 2 μl 10× buffer, 0.5 μl Pvu1, 0.5 μl ScaI, 1 μl H₂O. Both digests were incubated at 37° C. for 3 hours. Both the digested PCR fragment and the digested vector DNA were purified from 0.8% TAE agarose gels (FIG. 1A).

The PCR product was then ligated into Cst-04 vector. The ligation reaction contained 7 μl gel-purified Kan^{r} gene (cut with SacI/XbaI), 1 μl gel-purified Cst-04 vector (cut with BarnHI/EcoRI), 1 μl 10× Ligation Buffer 1 μl T4 DNA ligase, and was incubated on ice overnight. The ligated PCR product was then transformed into the TG1 competent cells. The transformation reaction conditions included 500 μl (thawed on ice) TG1 Competent cells and 5 μl pGEX-KT-exT-kanr- CNS-CstI ligation rxn. The cell/DNA mixture was incubated on ice for 30 minutes, and the cells were heat shocked at 42° C. for 45 seconds and then incubated on ice again for 2 minutes. 500 μl LB broth was added and the mixture was shaken at 37° C. for 1 hour. The transformation reactions were then plated on LB Kan^{r} plates and incubated 37° C. overnight. The results of the transformation reactions are shown in FIG. 1B.

Positive clones were screened for using the following method. Two milliliters of 9× LB/Kanamycin (10 g/ml) culture was incubated with individual transformants at 37° C. o/n using 250 RPM shaking. 1.5 milliliters of the overnight culture was transferred to an eppendorf tube to isolate plasmid DNA using Wizard Mini-Prep Kit (Qiagen, Valencia, Calif.).

An insert-containing colony (Cst-04-Kan5, FIG. 1C) was expanded in 100 ml of LB culture in order to isolate more plasmid DNA.

The Cst-04-Kan5 plasmid-containing colony was screened for kanamycin and ampicillin resistance. Cst-04-Kan5 colony was streaked on both AFLB Kan^{r} and Amp^{r} plates, which were incubated overnight at 37° C. FIG. 1D shows that, in colony Cst-04Kan5, the kanamycin resistance gene is active and the Ampicillin resistance gene is inactive.

The Cst-04Kan5 plasmid was transfomed into E. coli JM109 cells using 20 μl JM109 competent cells in 0.34 μl 1.42 M beta mercaptoethanol, incubated on ice for 10 minutes, then adding 1 μl (100 ng) Cst-04-Kan5 plasmid. The cell/DNA mixture was incubated ice for 30 minutes, then heat shocked at 42° C. for 45 seconds. The reaction was then incubated on ice for 2 minutes, at which time 80 μl SOC or LB was added. The reaction mixture was shaken at 37° C. for 1 hour, then plated on LB Kan^{r} plates. Identification and confirmation of the Cst-04-Kan5 plasmid DNA was carried out with a restriction enzyme digestion of plasmid DNA isolated from positive transformants, using NdeI, SalI, PstI restriction enzymes. The restriction fragment sizes were −7.3 kb for one cut, such as NdeI or SalI. Three bands (˜1.7 kb, ˜2.2 kb, and ˜3.2 kb) were observed when Cst-04-Kan5 DNA was cut with PstI (See FIG. 1C, lane 4).

Starter cultures of Cst-04Kan5 plasmid-containing cells were produced and used to inoculate 100 ml cultures for the generation of cell lysates. Centrifuged cell pellets resulting from the large-scale cultures were resuspended in 5 ml H₂0 prior to lysis in a French press. The resultant lysate was centrifuged at 4° C., 18,000 RPM for 20 minutes. The clarified lysate was subsequently used for activity analysis and the remainder of the lysate was stored at −20° C.

The activity of the cell lysates was determined under the assay conditions illustrated in Table 1.

TABLE 1

| | Final | Stock | | |
|---|---|---|---|---|
| Reagent | Concentration | Concentration | | Amounts |
| CTP | 1 mM | 100 | mM | 1 μl |
| NAN | 1 mM | 200 | mM | 0.5 μl |
| LacPsy | 0.5 mM | 2.5 | mM | 20 μl |
| MgCl2 | 50 mM | 1 | M | 5 μl |
| Tris pH 8 | 100 mM | 1 | M | 10 μl |

| | | | |
|---|---|---|---|
| Lysate | 15% | Crude | 15 μl |
| dH2O | | | 63 μl |
| Total | | | 100 μl |
| reaction | | | |
| volume | | | |

Table 1 lists the reagents, and concentrations and volumes thereof, used in the lysate activity assays.

The lysate assay reagents were mixed and incubated at 37° C. Time points were taken at 0 minutes and 1 hour. A negative control (pGEX-Kt-exT-kan^{r} vector without insert) was also included. All time points were analyzed using thin layer chromatography (CHCl₃:CH₃OH:H₂0:NH₄OH:60:40:5:1 respectively). The plates were air dried, dipped in anisaldehyde and heated on a hot plate until the spots developed. Cst-04Kan5 lysate was also assayed for activity using lacto-N-neotetraose as substrate. The lacto-N-neotetraose substrate activity is illustrated in FIG. 1E. Activity of lysates from Cst-04Kan5 plasmid-containing cells was 8500 units/liter.

### Example 2

### Modification of the Polylinker of pCWori+Kan^{r} Expression Vector

pCWori+Kan^{r} (Cst-04Kan5) contains the genes encoding N. meningitidis CMP-NAN synthetase (CNS) and Campylobacter jejuni α2,3 Sialyl Transferase (CstI) at the multiple cloning site. This vector appears to give high levels of expression of recombinant proteins but is hard to use due to the limited number of restriction sites in the multiple cloning site (MCS). Therefore, the multiple cloning site was modified and expanded as described herein.

The multiple cloning site starting at NdeI restriction site and extending to the start of the inactive Amp^{r} gene from CstO4Kan5 was generated using PCR to generate cDNA with modified multiple cloning sites at 5′ (Pcwmcs (NdeI)-ATCGATCGACATATGGGATCCGAGCTCAAGCTTTCTAGACTCGAGGAATTCGGTA CCGTCGACATCGATGATAAGCTGTCAAA) (SEQ ID NO:6) and 3′ (Scal-ATTCCAAGTAGTACTACTCTTCCTTTTTCAA) (SEQ ID NO:7) ends of pCWIN1 construct. The PCR primers set for pre pCWIN2 construct are 5′ (Bgl II-CAATTATATAGATCTATCGATGCTTAGGAGGT) (SEQ ID NO:8) and 3′ (Cstl-Xba-TTGCCTTATTCTAGATCATTAGTGGTGATGGTGGTG) (SEQ ID NO:9). The PCR products were then subcloned into Cst04kan5 (pCWori+kanr_CNS-CstI) vector, transformed into TG1 cells, and screened for the correct construct.

TwoPCR reactions were conducted, using 10 ng Cst04Kan5 cDNA as a template. The first reaction contained 1 μg (1 μl) Pcwmcs/ScaI-pcw primer, 78 II H₂O, 8 μl dNTP mixture, 10 μl 10× buffer, and 1 μl Vent polymerase. The second reaction contained 1 μg (1 μl) 5′pcBglII/CstI-Xba primer, 78 μl H₂O, 8 μl dNTP mixture, 10 μl 10× buffer, and 1 μl Vent polymerase. The PCR reaction parameters included a 5 minute cycle at 95° C., followed by the addition of 1 μl of Vent polymerase and thirty cycles of the following temperature pattern: 94° C. for one minute, 55° C. for one minute, 72° C. for one minute.

The PCR products were subjected to a restriction digest. The first PCR reaction product (“pCWIN1” insert) digest included 16 μl PCR r×n, 2 μl 10× buffer, 0.5 μl NdeI, 0.5 ,l Scal, 1 μl H₂O, and the second PCR reaction product (“pre pCWIN2” insert) digest included 16 μl PCR r×n, 2 μl 10× buffer, 0.5 μl BglII, 0.5 μl EcoRI, 1 μl H₂O. A pCWori Kan^{r} Cst04Kan5 vector was prepared for insertion of the first PCR product by incubation of 2 μl (1 μg) Cst04Kan5 vector, 2 μl 10× buffer, 0.5 μl NdeI, 0.5 μl ScaI, 1 μl H₂O. A pCWori Kan^{r} Cst04Kan5 vector was similarly prepared for insertion of the second PCR product by incubation of 2 μl (1 μg) Cst04Kan5 vector, 2 μl 10× buffer, 0.5 μl BamHI, 0.5 μI EcoRI, 1 μl H₂O.

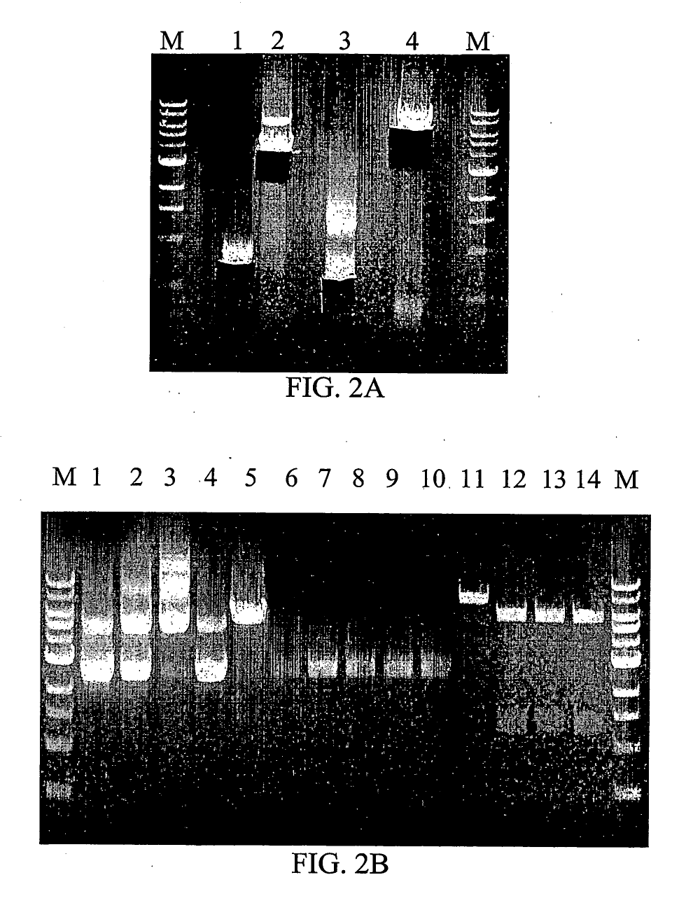

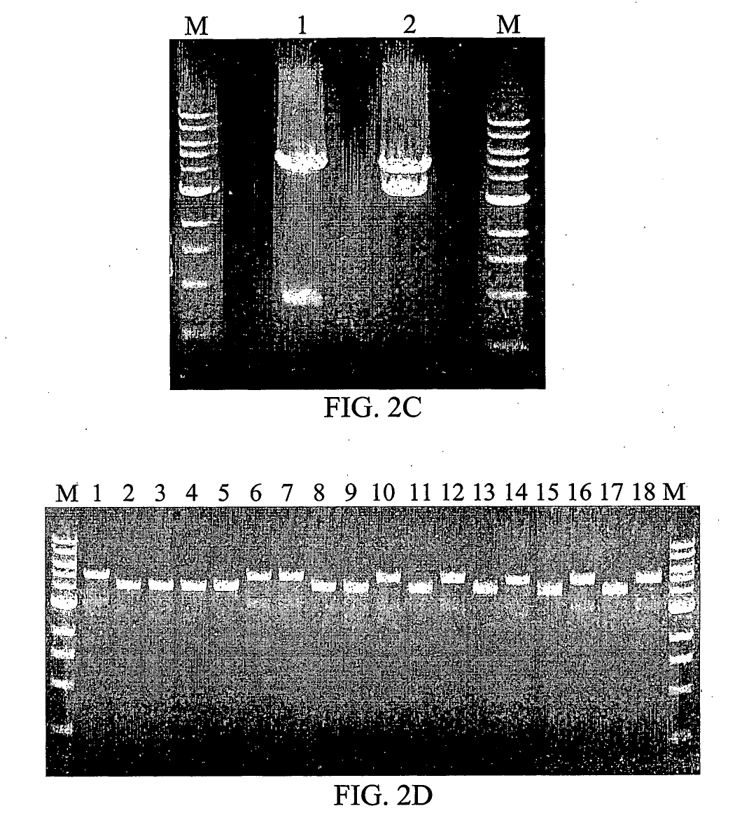

The digested PCR fragments and digested vectors were purified from 0.8% TAE agarose gels (FIG. 2A). The PCR products were then subcloned into Cst-04kan5 vectors by ligation (Table 2) and electroporated into the TG1 competent cells. Electroporation reactions included 30 μl thawed (on ice) TG1/DH5α electrocompetent cells and 3 μl ligation reaction mixture. The DNA/cell electroporation mixture was transferred to a chilled cuvette, and the cells were subjected to electroporation using pulses of 2.5 KV, R5 resistance, and 129 ohms. 0.9 ml SOC media was then added to the reaction mixture, and the entire culture was incubated at 37° C. for one hour, at which time the electoporation product was incubated overnight after plating on LB agar plates containing 50 μg/ml Kan^{r}.

TABLE 2

| | |
|---|---|
| 1. pCWIN1 | |
| Gel-purified pCWIN1 insert (digested A) | 7 μl |
| Gel-purified Cst-04kan5 vector (digested B) | 1 μl |
| 10X Ligation Buffer | 1 μl |
| T4 DNA Ligase | 1 μl |
| 2. pre pCWIN2 | |
| Gel-purified pre pCWIN2 insert (digested C) | 7 μl |
| Gel-purified Cst-04kan5 vector (digested D) | 1 μl |
| 10X Ligation Buffer | 1 μl |
| T4 DNA Ligase | 1 μl |
| 3. pCWIN2 | |
| Gel-purified pCW1N1#5 insert (digested E) | 7 μl |
| Gel-purified pre pCWIN2#11 vector (digested F) | 1 μl |
| 10X Ligation Buffer | 1 μl |
| T4 DNA Ligase | 1 μl |

Table 2 illustrates the ligation reaction conditions for pCWIN1, pre-pCWIN2, and pCWIN2 PCR reaction products. Both pCWIN1 and pre pCWIN2 ligations were incubated at 4° C. overnight.

Screening of transformants for positive clones was then conducted. Five colonies pCWIN1-containing TG1 cells were selected, as were 9 colonies of pre-pCWin2 in DH5α, and 18 colonies of pCWin2 in TG1. Each was placed into 2 ml TB/kanamycin (50 μg/ml) and incubated at 37° C. for 5 hours with shaking at 250 RPM. 1.5 milliliters of each culture was transferred to an eppendorf tube to isolate plasmid DNA using Wizard Plus Mini-Prep Kit (Qiagen, Valencia, Calif.). Each plasmid DNA preparation was subjected to restriction digestion with the appropriate restriction endonucleases as described above. The digestion reactions were then analyzed on agarose/TAE gels (FIGS. 2B-2D).

One colony of pCWin1 (colony #5) and one colony of pre pCWin2 (colony #11) contained the appropriate sized inserts. Specifically, the expected size for the pCWinl insert was 5 kb and the expected size for the pre-pCWin2 insert was 7 kb (FIG. 2B). pCWin1 and pre pCWin2 plasmid DNA were digested with NdeI and Scal. NdeI and ScaI digestion of pCWin1 plasmid generated bands of 750 bp and 4.2 kb, and the same digestion of pre-pCWin2 plasmid generated bands of 2.8 kb and 4.2 kb (FIG. 2C). FIG. 2D illustrates the result of ligating the 720 bp of pCWin1 with the 4.2 kb fragment of pre-pCWin2 to generate the pCWin2 expression vector. The difference between pCWinl and pCWin2 expression is that pCWin1 has two BamH1 sites, one in being in the tac promoter and the other being in the multiple cloning site (down stream of NdeI). pCWin2 has only one BamH1 site, in the multiple cloning site, and the BamH1 that resided in the tac promoter was destroyed.

### Example 3

### Addition of Maltose Binding Protein to pCWin2 Kan^{r} Expression Vector

The E.coli ma1E gene, encoding a maltose binding protein, was subcloned into the pCWin2 kan^{r} bacterial expression vector. The ma1E gene was PCR amplified from pMal-c2X, ligated into the multiple cloning site of pCWin2 kanr, and subsequently transformed into electrocompetent DH5a E. coli. The final product, a pCWin2MBP kan^{r} bacterial fusion tag expression vector, was created as described below.

Restriction endonuclease digestion of pCWin2 kan^{r} and pMAL-c2X amp^{r} was conducted to prepare the ma1E maltose binding protein cDNA and the pCWin2 vector cDNA for insertion of the ma1E cDNA into the pCWin2 vector. Digestion of the ma1E cDNA was conducted using 2 μl of pMAL -C2X vector DNA (1 μg/μl), 2 μl 10× BamHI NEbuffer, 2 μl 10× purified BSA, 1 μl NdeI, 1 μl BamHI, and 12 μl dH₂O. Digestion of the vector was conducted using 2 μl pCWin2 vector DNA 0.8 μg/μl, 2 ,μl 10× BamHI NEbuffer, 2 μl 10× purified BSA, 1 μl NdeI, 1 μl BamHI, and 12 μl dH₂O.

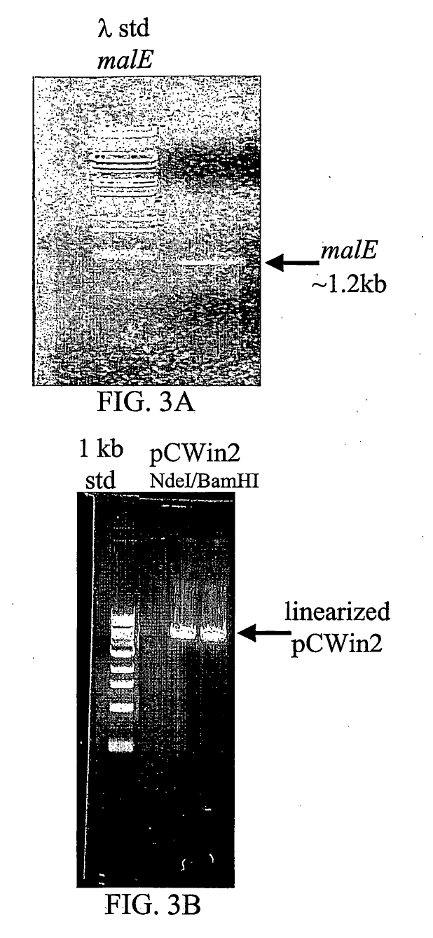

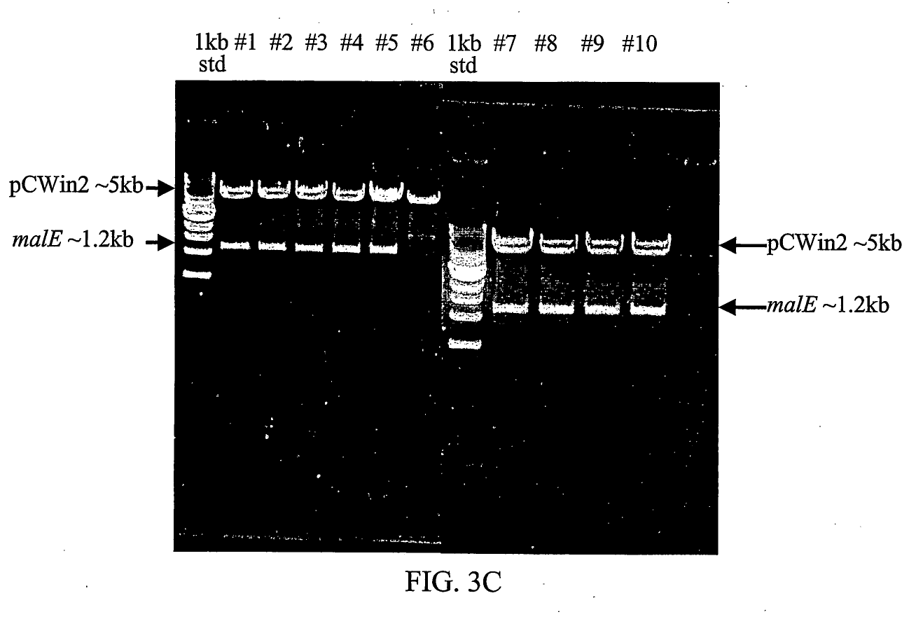

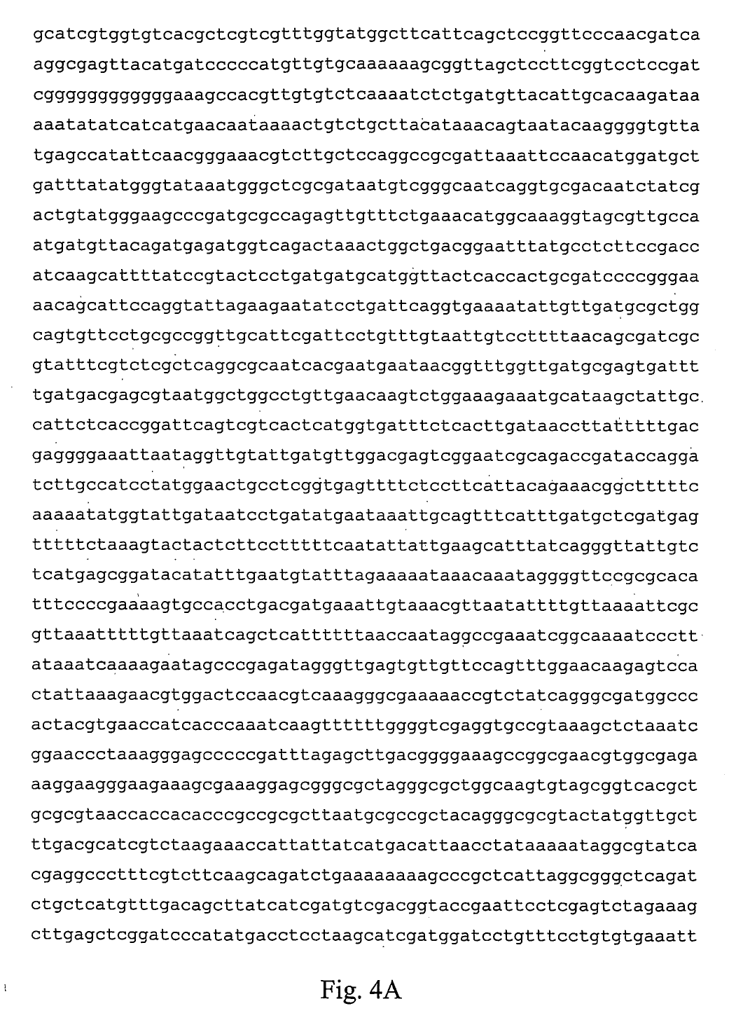

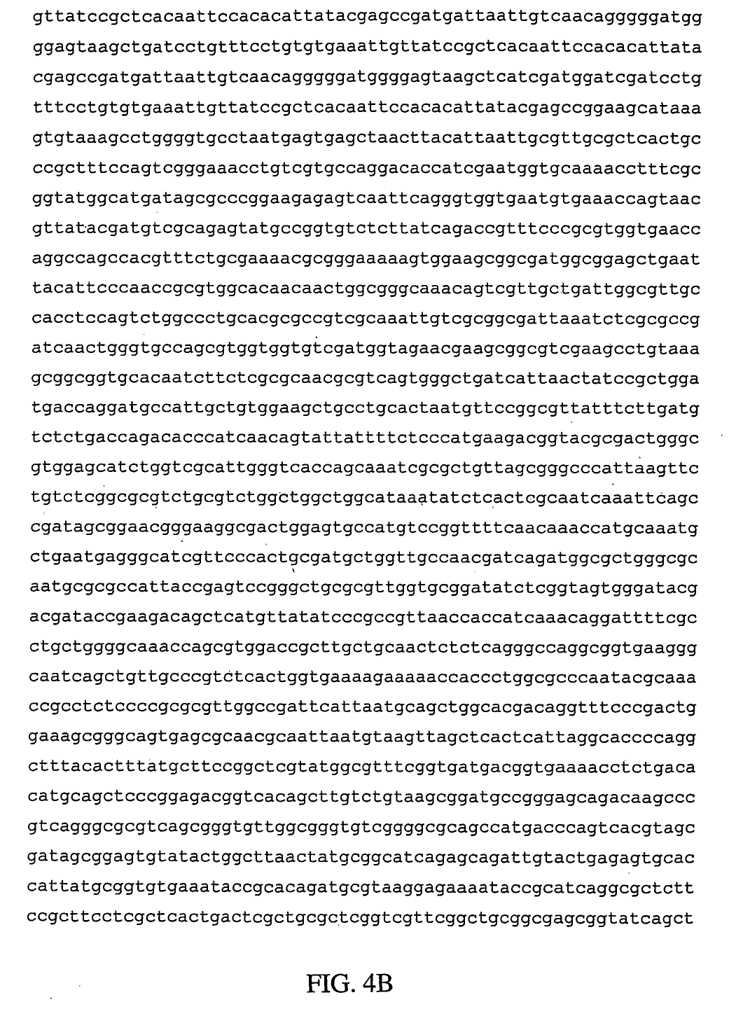

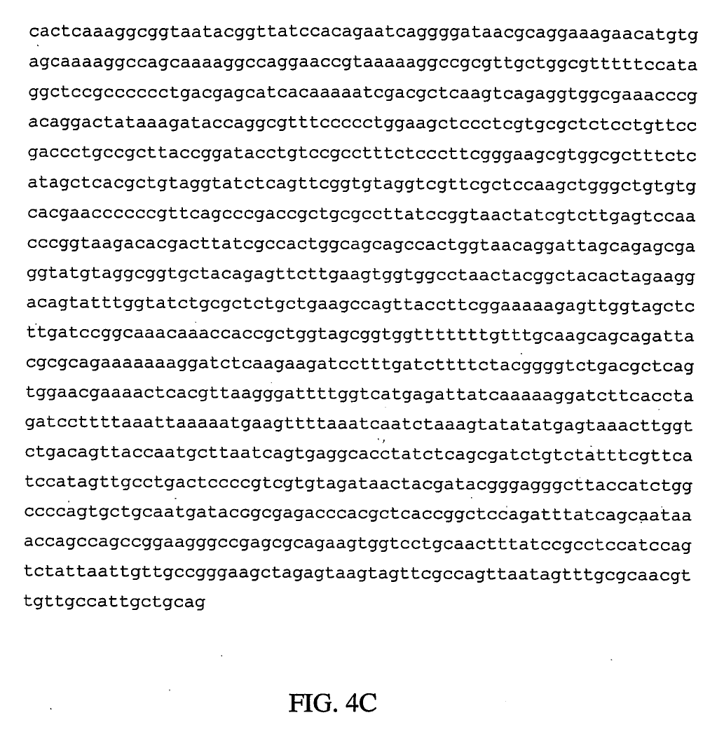

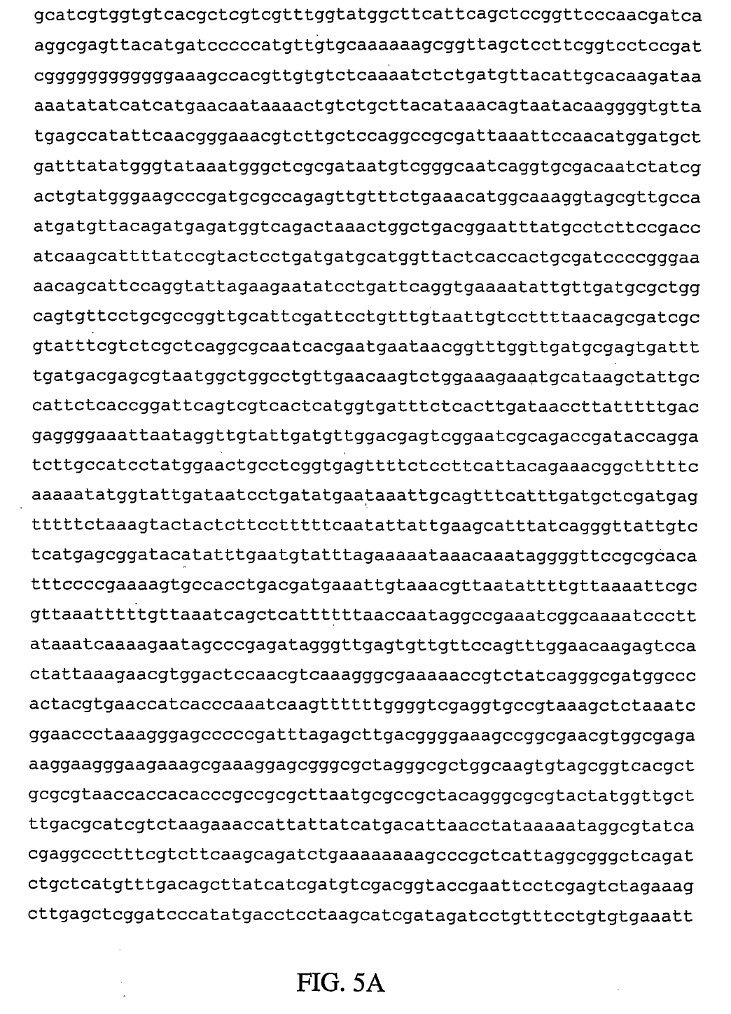

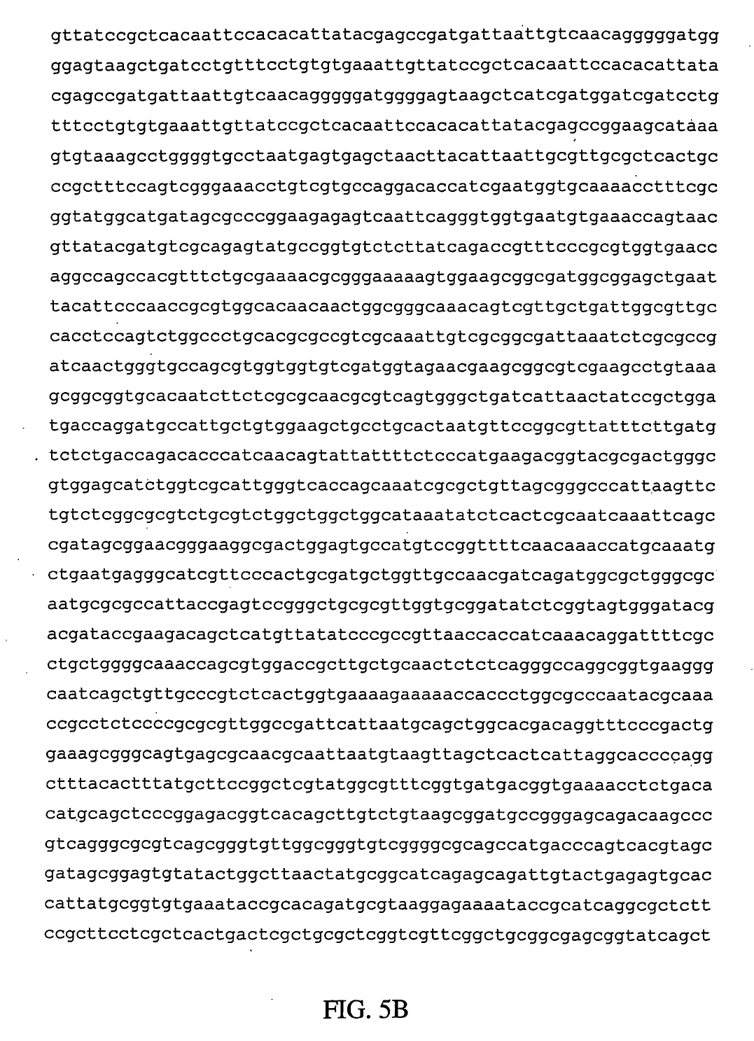

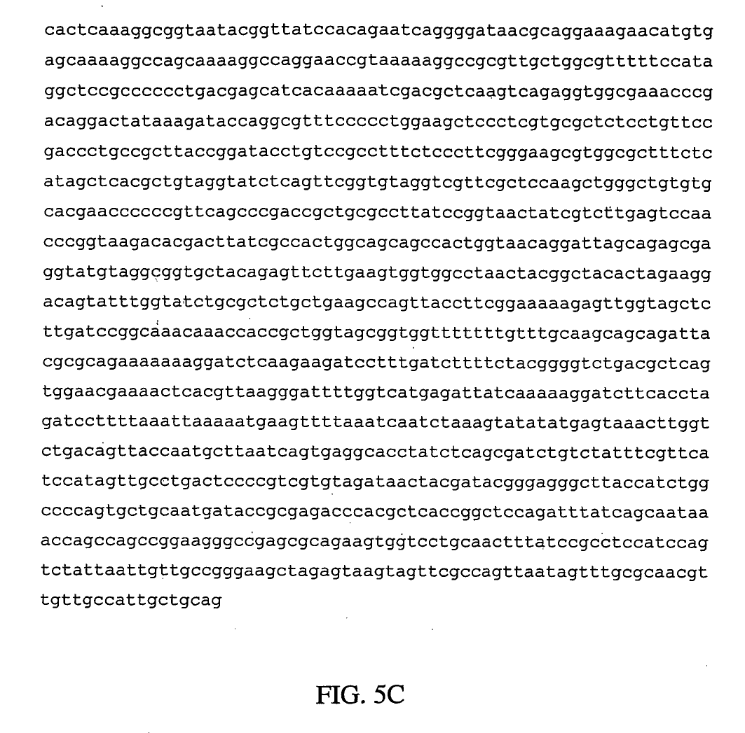

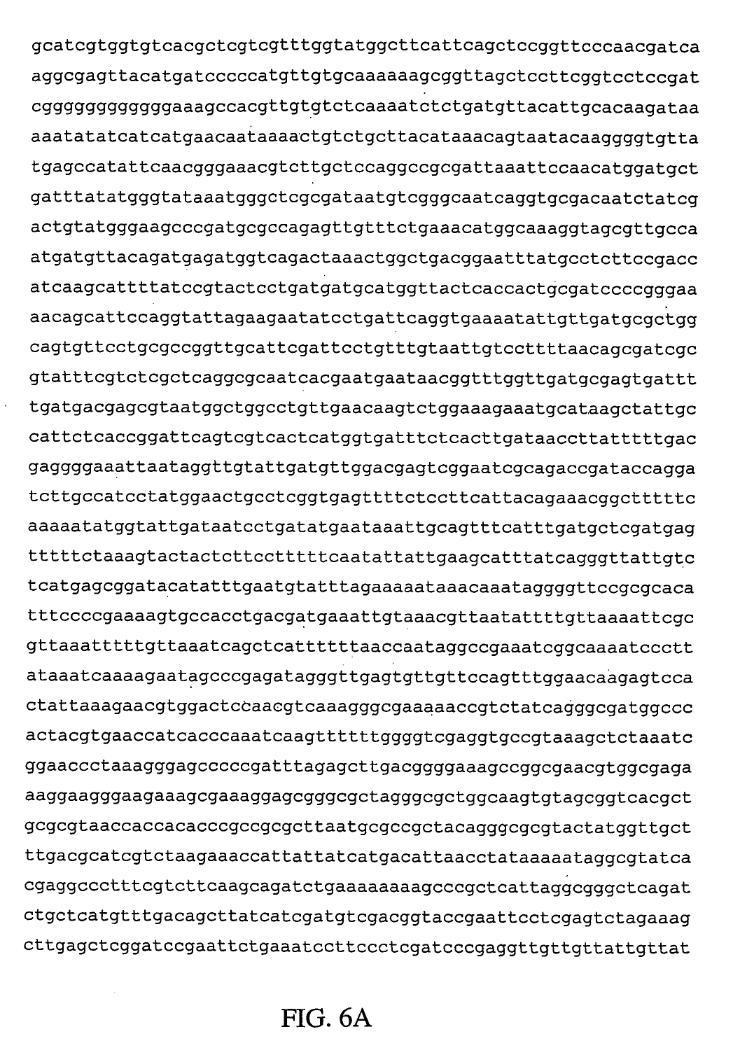

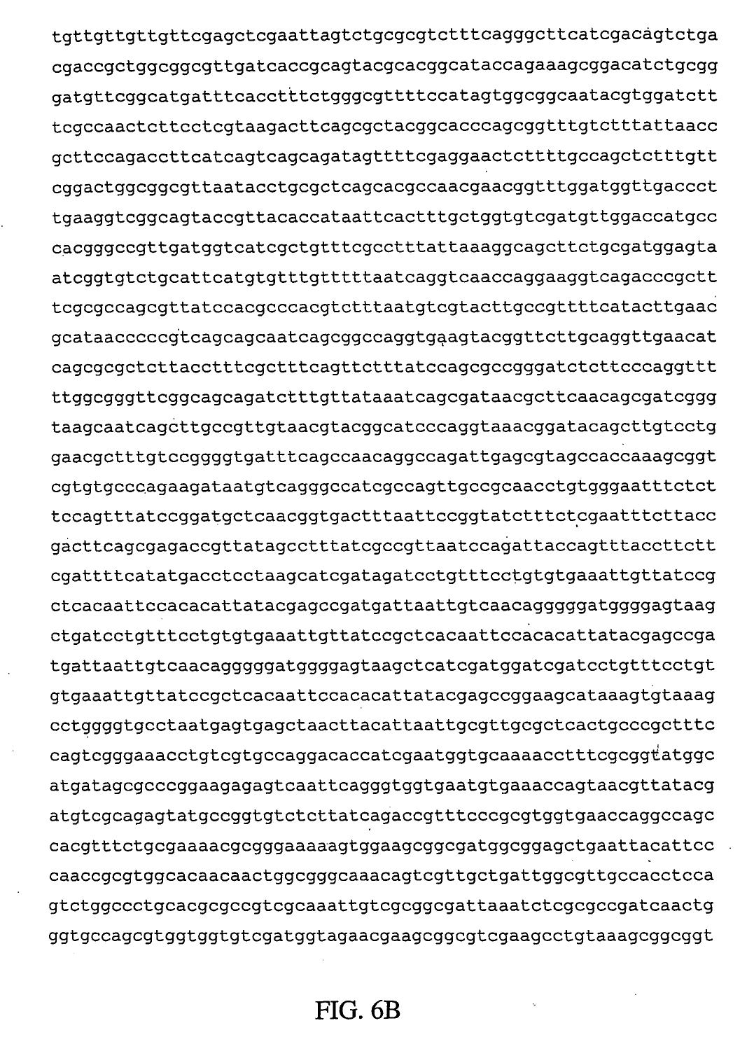

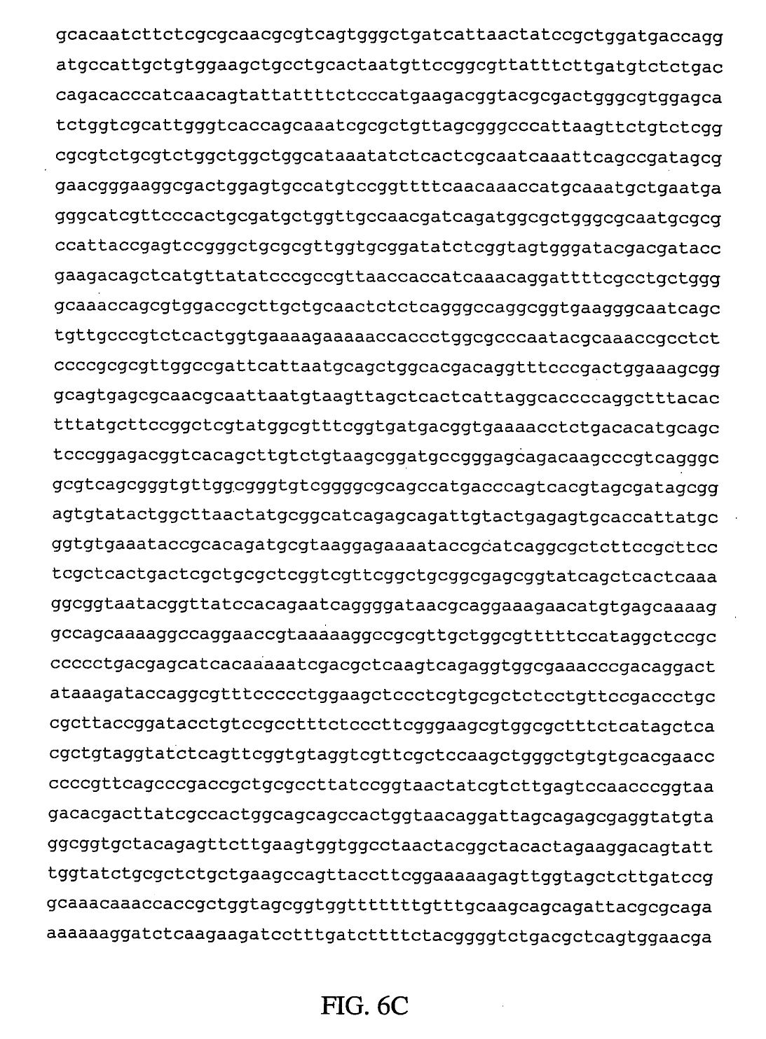

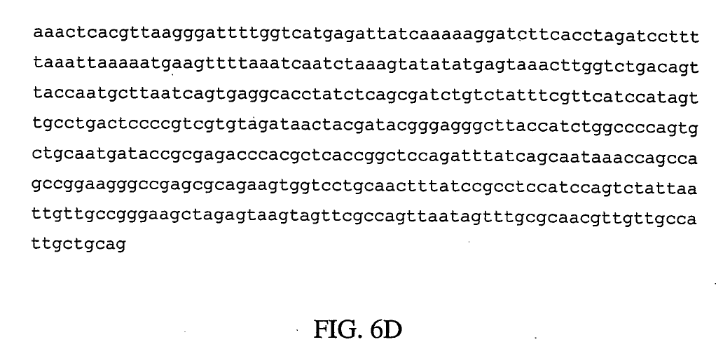

The restriction enzyme digestions were incubated at 37° C. for two hours. The reactions were stopped by adding 3 μl Blue/Orange 6× Agarose Loading Dye. The digestions were then loaded onto separate 0.7% agarose/TAE gels, and electrophoresed at 135 volts until the dyes migrated to the lower third of the gel. An image of the pCWin2 vector digestion agarose gel was captured using a digital camera (FIG. 3B). An image of the polyacrylamide gel containing the purified product from the digestion of ma1E is shown in FIG. 3A.

The linearized pCWin2 kan^{r} and ma1E fragments were gel purified. Using the UV box to illuminate the DNA, the bands of DNA were excised using a sterile scalpel. The pCWin2 kan^{r} DNA was approximately 5 kb, and the ma1E gene was approximately 1.2 kb. The excised agarose wedges were placed into Ultrafree DA agarose extraction spinfilters (Millipore, Bellerica, Mass.), and microcentrifuged at 10,000 ×g for 5 minutes. The filtrates were transferred to YM-100 spinfilters (Millipore, Bellirica, Mass.), and the DNA was washed by adding 300 μl dH₂O. The spinfilters were centrifuged at 500×g for 15 minutes, and the wash step was repeated two additional times. The last wash step concentrated the DNA to an approximate volume of 25 μl, at which time the column was inverted into another autoclaved 1.7 mL eppendorf tube. The DNA retentate was collected by microcentrifuging the eppendorf at 1000×g for one minute.

The ligation of gel purified ma1E and linearized pCWin2 kan^{r} was performed in an autoclaved 0.5 mL eppendorf microcentriuge tube. The ligation reaction included 7 μl of purified ma1E DNA that was digested with NdeI and BamHI, 1 μl of linearized pCWin2 kan^{r} that was digested with NdeI and BamHI, 1 μl 10× ligase buffer, 1 μl T4 DNA ligase. The ligation reaction was incubated at room temperature for three hours. In the vector control ligation reaction, dH₂0 was substituted for the ma1E DNA.

The ligation reactions were transformed into electrocompetent DH5a E.coli. After a three hour ligation incubation, one aliquot of electrocompetent DH5a E.coli was removed from a −81° C. freezer, and placed on ice to thaw. 20 μl of the cells was aliqouted into chilled, autoclaved 1.7 mL microcentrifuge tubes, and then one microliter from each ligation reaction was added to the cells. Immediately, the reactions were transferred to chilled electroporation cuvettes. The cells were electroporated with a 2.5 kV 6 msec pulse as described in the manufacturer's instructions. Then one milliliter of AFLB SOC media was added to the transformation reactions, and the entire volume was transferred to an autoclaved 1.7 mL microcentrifuge tube. The transformation reactions were incubated at 37° C. for one hour with shaking at 250 rpm. After incubating the cells for an hour, 100 μl from each transformation reaction was plated by spreading onto LB agar kanrplates. The plates were incubated at 37° C. overnight.

Results from the ligation and transformation reactions are as follows. The pCWin2 MBP vector plating resulted in thirteen colonies, and out of ten colonies selected, nine were positive for the recombinant vector. The pCWin2 vector control plating did not contain any colonies. Colonies from the pCWin2 MBP vector LB agar plates were selected and used to inoculate 2 ml AFLB kan^{r}. The starter cultures were grown overnight at 37° C., with shaking at 250 rpm. Plasmid DNA was isolated from the transformants and screened for the correct ma1E insert by a double digestion with NdeI and BamHI restriction endonucleases.

Restriction digestion of miniprep DNA was conducted in a reaction mixture containing 12 μl Miniprep DNA, 1 μl NdeI endonuclease, 1 μl BamHI endonuclease, 1.5 μl I XBamHI NEBuffer, and 1 μl 10× purified BSA. The digestion reactions were mixed and incubated at 37° C. for one hour. After incubation, 3 μl of 6× Agarose Gel Loading Dye was added to each restriction digestion. The restriction digestions were then loaded into the wells of a 0.7% agarose/TAE gel. The samples were electrophoresed at 135 volts until the dye migrated to the lower third of the gel. The gel was then removed from the gel box, and the image captured with a digital camera (FIG. 3C).

Large scale purified pCWin2MBP vector DNA was isolated from transformant #1 using the HiSpeed Plasmid Maxi Kit (Qiagen, Valencia, Calif.). A 2 mL AFLB kan^{r} starter culture was inoculated with 10 μl pCWin2MBP DH5a E.coli overnight culture. This starter culture was grown overnight in a 37° C. incubator, with shaking at 250 rpm. The overnight starter culture was used to inoculate two 125 mL AFLB kan^{r} cultures, and these larger scale preps were grown overnight at 37° C. with shaking at 250 rpm. DNA from the large scale preparation was used for sequencing of the ma1E insert subcloned between the NdeI and BamHI restriction sites (MWG Biotech, High Point, N.C.). The sequence of the vector is set forth in SEQ ID NO:3.

### Example 4

### Preparation and Characterization of pCWin2-MBP-SBD₃₉(LMS₃₉) Vector

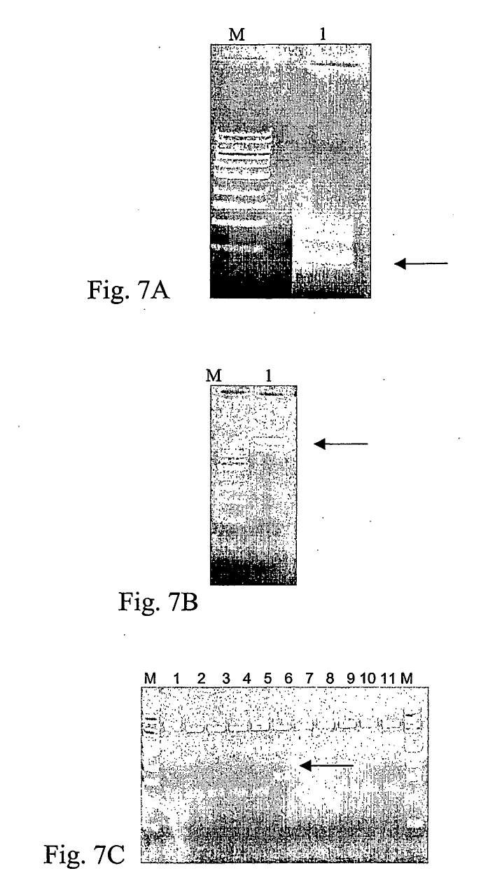

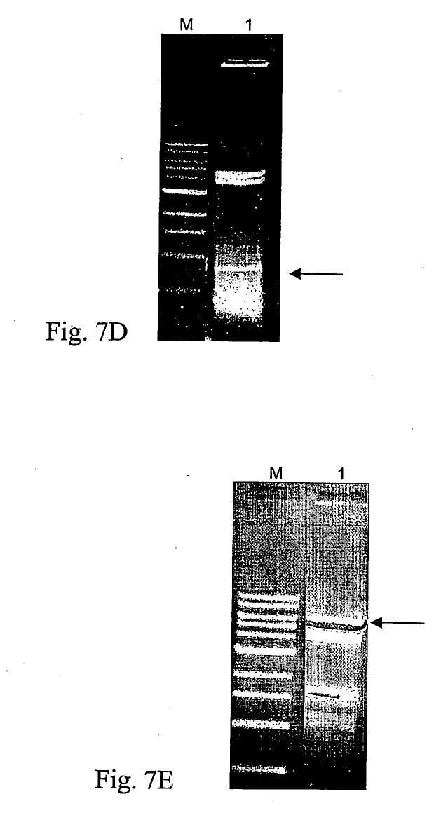

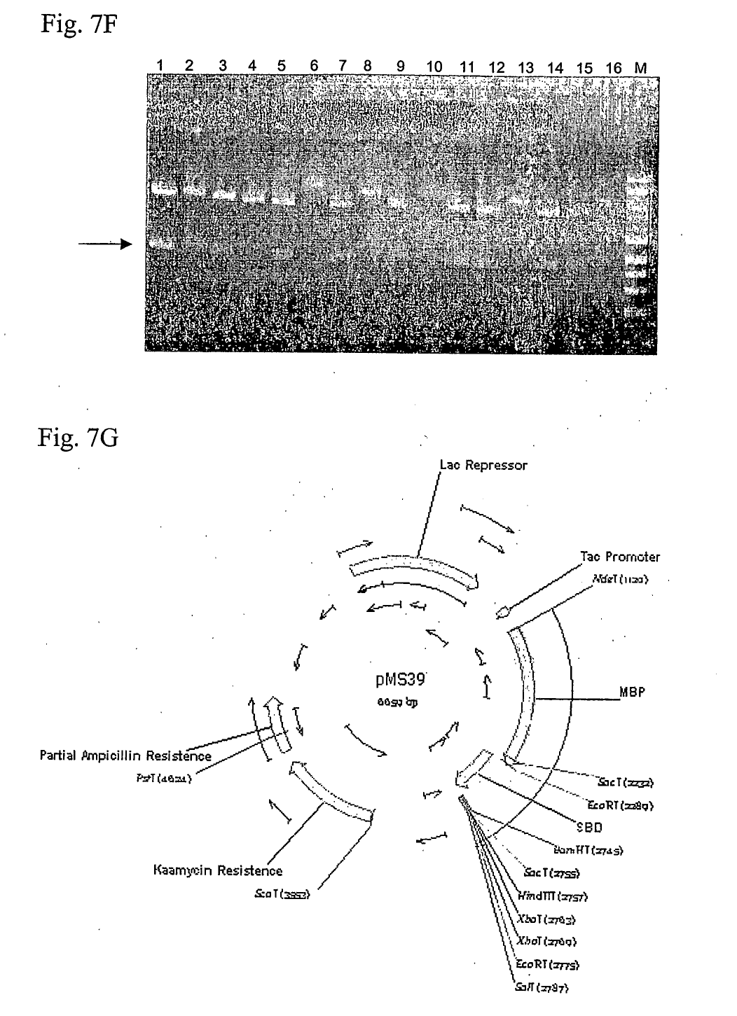

The pMS₃₉ Kan^{R} expression vector was created from the pCWIN2-MBP-SBD-ST3 Gal III (Galβ1,3(4)GlcNAc a2,3-Sialyltransferase) Δ73 construct, removing the the ST3 Gal III Δ73 gene and replacing it with the Multiple Cloning site (MCS) from the pcWIN2 vector. Selection was of final construct was determined by restriction enzyme analysis with Nde I and Xba I (there is no Xba I site the pCWIN2-MBP-SBD ST3 Gal III construct) digestion and sequence confirmation. The final construct was designated the pCWin2-MBP-SBD (PMS₃₉) Kan^{r} expression vector. The several steps of the preparation of this vector are detailed in FIGS. 7A-7G

### Example 5

### Preparation and Characterization of the pCWin2-MBP-MCS-SBD (I)MXSL9) Vector

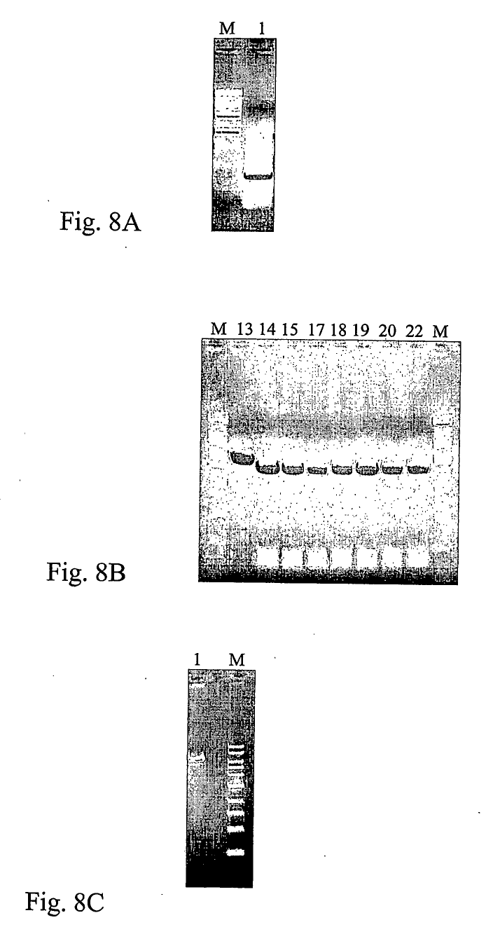

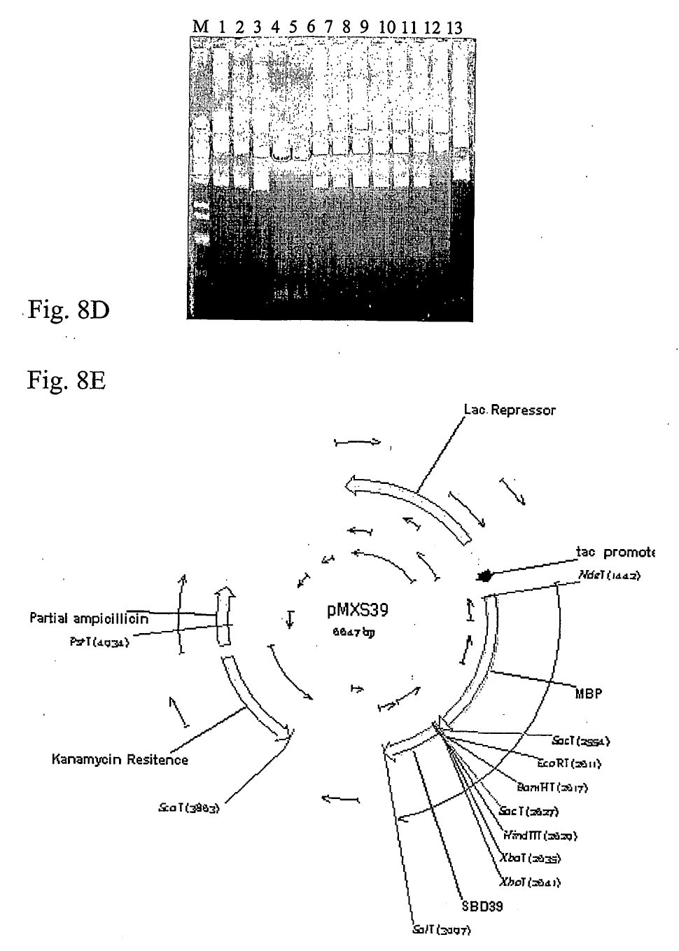

The pCWin2-MBP-MCS-SBD (PMXS₃₉) expression vector was constructed according to the following method. The Starch Binding Domain (SBD) insert was isolated by PCR using the 5′ primer ( XhoI-SBD-39-5′ TGTATCCTCGAGATTGTGGCGACCGGCGGCACCAC) (SEQ ID NO:12) and the 3′ primer (3′ SalI-CTTGTCGACTCATTAGCGCCAGGTATCGGTCACGG) (SEQ ID NO:13). The PCR products were gel purified and ligated into PCR-Blunt vector. The correct SBD insert (in the PCR-Blunt vector) was digested with Xho1 and Sal1, subdloned into Xho1-Sal1 digested pCWin2-MBP kan^{r} vector, transformed into TB1 cells and screened for the correct construct. The several steps of the preparation of this vector are detailed in FIGS. 8A-8E.

The disclosures of each and every patent, patent application, and publication cited herein are hereby incorporated herein by reference in their entirety. While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of providing a therapeutic protein to a customer, said method comprising cloning a nucleic acid encoding said protein into a pCWinl expression vector as set forth in SEQ ID NO:1, expressing said protein therefrom, and providing said protein to said customer.

2. A method of providing a therapeutic protein to a customer, said method comprising cloning a nucleic acid encoding said protein into a pCWin2 expression vector as set forth in SEQ ID NO:2, expressing said protein therefrom, and providing said protein to said customer.

3. A method of providing a therapeutic protein to a customer, said method comprising cloning a nucleic acid encoding said protein into a nucleic acid vector selected from the group consisting of:
a) a pCWin2/MBP expression vector as set forth in SEQ ID NO:3;
b) a pCWin2-MBP-SBD (pMS₃₉) expression vector as set forth in SEQ ID NO:10; and
c) a pCWin2-MBP-MCS-SBD (pMXSp₃₉) expression vector as set forth in SEQ ID NO:11;
expressing said protein therefrom, and providing said protein to said customer.

4. The method of claim 3, wherein said nucleic acid vector comprises a protease cleavage site coding sequence at a location selected from the group consisting of:
a) between the MBP coding sequence and the therapeutic protein coding sequence; and
b) immediately prior to the start of the C-terminus of the MBP coding sequence.

5. The method of claim 2 or 3, wherein said protein is selected from the group consisting of erythropoietin, human growth hormone, granulocyte colony stimulating factor, interferons alpha, -beta, and -gamma, Factor IX, follicle stimulating hormone, interleukin-2, erythropoietin, anti-TNF-alpha, and a lysosomal hydrolase.

6. The method of claim 5, wherein said lysosomal hydrolase is selected from the group consisting of beta-glucosidase, alpha-galactosidase-A, beta-hexosaminidase, beta-galactosidase, alpha-galactosidase, alpha-mannosidase, beta-mannosidase, alpha-L-fucosidase, beta-glucuronidase, alpha-glucosidase, alpha-N-acetylgalactosaminidase, and acid phosphatase.

7. A method of providing a protein to a customer, said method comprising cloning a nucleic acid encoding said protein into a pCWin1 expression vector as set forth in SEQ ID NO:1, expressing said protein therefrom, and providing said protein to said customer.

8. A method of providing a protein to a customer, said method comprising cloning a nucleic acid encoding said protein into a pCWin2 expression vector as set forth in SEQ ID NO:2, expressing said protein therefrom, and providing said protein to said customer.

9. A method of providing a protein to a customer, said method comprising cloning a nucleic acid encoding said protein into a nucleic acid vector selected from the group consisting of:
a) a pCWin2/MBP expression vector as set forth in SEQ ID NO:3;
b) a pCWin2-MBP-SBD (pMS₃₉) expression vector as set forth in SEQ ID NO:10; and
c) a pCWin2-MBP-MCS-SBD (pMXS₃₉) expression vector as set forth in SEQ ID NO:11;
expressing said protein therefrom, and providing said protein to said customer.

10. The method of claim 7, 8 or 9, wherein said protein is selected from the group consisting of a glycosyltransferase and a sugar nucleotide-generating enzyme.

11. A method of providing a protein to a customer, said method comprising providing a pCWin1 vector as set forth in SEQ ID NO:1 to a protein production facility, wherein a nucleic acid encoding said protein is cloned into said expression vector and said protein is expressed therefrom in said protein production facility, and providing said protein to said customer.

12. A method of providing a protein to a customer, said method comprising providing a pCWin2 vector as set forth in SEQ ID NO:2 to a protein production facility, wherein a nucleic acid encoding said protein is cloned into said expression vector and said protein is expressed therefrom in said protein production facility, and providing said protein to said customer.

13. A method of providing a protein to a customer, said method comprising providing a nucleic acid vector selected from the group consisting of:
a) a pCWin2/MBP expression vector as set forth in SEQ ID NO:3;
b) a pCWin2-MBP-SBD (pMS₃₉) expression vector as set forth in SEQ ID NO:10; and
c) a pCWin2-MBP-MCS-SBD (pMXS₃₉) expression vector as set forth in SEQ ID NO:11;
to a protein production facility, wherein a nucleic acid encoding said protein is cloned into said expression vector and said protein is expressed therefrom in said protein production facility, and providing said protein to said customer.

14. The method of claim 2, 3, 4, 7, 8 or 9, wherein said method further comprises prior to providing said protein to said customer, at least one glycosyl moiety is added to said protein.

15. The method of claim 14, wherein said glycosyl moiety is added to said protein in vitro.

16. A method of providing a protein to a customer, said method comprising cloning a nucleic acid encoding said protein into-nucleic acid vector selected from the group consisting of:
a) a pCWin1 vector as set forth in SEQ ID NO:1;
b) a pCWin2 vector as set forth in SEQ ID NO:2;
c) a pCWin2/MBP vector as set forth in SEQ ID NO:3;
d) a pCWin2-MBP-SBD (pMS₃₉) vector as set forth in SEQ ID NO:10; and
e) a pCWin2-MBP-MCS-SBD (pMXS₃₉) vector as set forth in SEQ ID NO:11;
further wherein said method comprises inserting said vector into a bacterial host cell, expressing said protein in said host cell, and providing said protein to said customer.

17. The method of claim 16, wherein said method further comprises prior to providing said protein to said customer, at least one glycosyl moiety is added to said protein.

18. The method of claim 16, wherein said glycosyl moiety is added to said protein in vitro.

19. The method of claim 16, wherein said expression vector further comprises an affinity tag coding sequence.

20. An isolated pcWIN1 expression vector comprising the sequence set forth in SEQ ID NO:1.

21. An isolated pcWIN1 expression vector consisting of the sequence set forth in SEQ ID NO:1.

22. An isolated pcWIN2 expression vector comprising the sequence set forth in SEQ ID NO:2.

23. An isolated pcWIN2 expression vector consisting of the sequence set forth in SEQ ID NO:2.

24. An isolated pcWIN2/MBP expression vector comprising the sequence set forth in SEQ ID NO:3.

25. An isolated pcWIN2/MBP expression vector consisting of the sequence set forth in SEQ ID NO:3.

26. The pcWIN280P expression vector of claim 24, wherein the pCWIN2/MBP vector comprises a protease cleavage site coding sequence adjacent to the MBP coding sequence.

27. An isolated pCWin2-MBP-SBD (pMS₃₉) vector comprising the sequence set forth in SEQ ID NO:10.

28. An isolated pCWin2-MBP-SBD (pMS₃₉) vector consisting of the sequence set forth in SEQ ID NO:10.

29. An isolated pCWin2-MBP-MCS-SBD (pMXS₃₉) vector comprising the sequence set forth in SEQ ID NO:11.

30. An isolated pCWin2-MBP-MCS-SBD (pMXS₃₉) vector consisting of the sequence set forth in SEQ ID NO:11.

31. The pCWin2-MBP-SBD (pMS₃₉) expression vector of claim 27, wherein the pCWin2-MBP-SBD (pMS₃₉) vector comprises a protease cleavage site coding sequence immediately prior to the start of the C-terminus of the MBP coding sequence.

32. A method of expressing a protein, said method comprising cloning a nucleic acid encoding said protein into a pCWin1 expression vector as set forth in SEQ ID NO:1 and expressing said protein therefrom.

33. A method of expressing a protein, said method comprising cloning a nucleic acid encoding said protein into a pCWin2 expression vector as set forth in SEQ ID NO:2 and expressing said protein therefrom.

34. A method of expressing a protein, said method comprising cloning a nucleic acid encoding said protein into a nucleic acid vector selected from the group consisting of:
a) a pCWin2/MBP expression vector as set forth in SEQ ID NO:3;
b) a pCWin2-MBP-SBD (pMS₃₉) expression vector as set forth in SEQ ID NO:10; and c) a pCWin2-MBP-MCS-SBD (pMXS₃₉) expression vector as set forth in SEQ ID NO:11;
and expressing said protein therefrom.

35. The method of any one of claims 32-34, wherein said protein is expressed in a prokaryotic cell.


SEQUENCE LISTING (13 sequences)

SEQ ID NO: 1 - DNA, 5039 bp, Artificial Sequence
  Custom DNA vector
gcatcgtggtgtcacgctcgtcgtttggtatggcttcattcagctccggttcccaacgatcaaggcgagttacatgatcccccatgttgtgcaaaaaagcggttagctccttcggtcctccgatcggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctccaggccgcgattaaattccaacatggatgctgatttatatgggtataaatgggctcgcgataatgtcgggcaatcaggtgcgacaatctatcgactgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctattgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaagtactactcttcctttttcaatattattgaagcatttatcagggttattgtctcatgagcggatacatatttgaatgtatttagaaaaataaacaaataggggttccgcgcacatttccccgaaaagtgccacctgacgatgaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgcatcgtctaagaaaccattattatcatgacattaacctataaaaataggcgtatcacgaggccctttcgtcttcaagcagatctgaaaaaaaagcccgctcattaggcgggctcagatctgctcatgtttgacagcttatcatcgatgtcgacggtaccgaattcctcgagtctagaaagcttgagctcggatcccatatgacctcctaagcatcgatggatcctgtttcctgtgtgaaattgttatccgctcacaattccacacattatacgagccgatgattaattgtcaacagggggatggggagtaagctgatcctgtttcctgtgtgaaattgttatccgctcacaattccacacattatacgagccgatgattaattgtcaacagggggatggggagtaagctcatcgatggatcgatcctgtttcctgtgtgaaattgttatccgctcacaattccacacattatacgagccggaagcataaagtgtaaagcctggggtgcctaatgagtgagctaacttacattaattgcgttgcgctcactgcccgctttccagtcgggaaacctgtcgtgccaggacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccccaggctttacactttatgcttccggctcgtatggcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccattatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcag

SEQ ID NO: 2 - DNA, 5039 bp, Artificial Sequence
  Custom DNA vector
gcatcgtggtgtcacgctcgtcgtttggtatggcttcattcagctccggttcccaacgatcaaggcgagttacatgatcccccatgttgtgcaaaaaagcggttagctccttcggtcctccgatcggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctccaggccgcgattaaattccaacatggatgctgatttatatgggtataaatgggctcgcgataatgtcgggcaatcaggtgcgacaatctatcgactgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctattgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaagtactactcttcctttttcaatattattgaagcatttatcagggttattgtctcatgagcggatacatatttgaatgtatttagaaaaataaacaaataggggttccgcgcacatttccccgaaaagtgccacctgacgatgaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgcatcgtctaagaaaccattattatcatgacattaacctataaaaataggcgtatcacgaggccctttcgtcttcaagcagatctgaaaaaaaagcccgctcattaggcgggctcagatctgctcatgtttgacagcttatcatcgatgtcgacggtaccgaattcctcgagtctagaaagcttgagctcggatcccatatgacctcctaagcatcgatagatcctgtttcctgtgtgaaattgttatccgctcacaattccacacattatacgagccgatgattaattgtcaacagggggatggggagtaagctgatcctgtttcctgtgtgaaattgttatccgctcacaattccacacattatacgagccgatgattaattgtcaacagggggatggggagtaagctcatcgatggatcgatcctgtttcctgtgtgaaattgttatccgctcacaattccacacattatacgagccggaagcataaagtgtaaagcctggggtgcctaatgagtgagctaacttacattaattgcgttgcgctcactgcccgctttccagtcgggaaacctgtcgtgccaggacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccccaggctttacactttatgcttccggctcgtatggcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccattatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcag

SEQ ID NO: 3 - DNA, 6209 bp, Artificial Sequence
  Custom DNA vector
gcatcgtggtgtcacgctcgtcgtttggtatggcttcattcagctccggttcccaacgatcaaggcgagttacatgatcccccatgttgtgcaaaaaagcggttagctccttcggtcctccgatcggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctccaggccgcgattaaattccaacatggatgctgatttatatgggtataaatgggctcgcgataatgtcgggcaatcaggtgcgacaatctatcgactgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctattgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaagtactactcttcctttttcaatattattgaagcatttatcagggttattgtctcatgagcggatacatatttgaatgtatttagaaaaataaacaaataggggttccgcgcacatttccccgaaaagtgccacctgacgatgaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgcatcgtctaagaaaccattattatcatgacattaacctataaaaataggcgtatcacgaggccctttcgtcttcaagcagatctgaaaaaaaagcccgctcattaggcgggctcagatctgctcatgtttgacagcttatcatcgatgtcgacggtaccgaattcctcgagtctagaaagcttgagctcggatccgaattctgaaatccttccctcgatcccgaggttgttgttattgttattgttgttgttgttcgagctcgaattagtctgcgcgtctttcagggcttcatcgacagtctgacgaccgctggcggcgttgatcaccgcagtacgcacggcataccagaaagcggacatctgcgggatgttcggcatgatttcacctttctgggcgttttccatagtggcggcaatacgtggatctttcgccaactcttcctcgtaagacttcagcgctacggcacccagcggtttgtctttattaaccgcttccagaccttcatcagtcagcagatagttttcgaggaactcttttgccagctctttgttcggactggcggcgttaatacctgcgctcagcacgccaacgaacggtttggatggttgacccttgaaggtcggcagtaccgttacaccataattcactttgctggtgtcgatgttggaccatgcccacgggccgttgatggtcatcgctgtttcgcctttattaaaggcagcttctgcgatggagtaatcggtgtctgcattcatgtgtttgtttttaatcaggtcaaccaggaaggtcagacccgctttcgcgccagcgttatccacgcccacgtctttaatgtcgtacttgccgttttcatacttgaacgcataacccccgtcagcagcaatcagcggccaggtgaagtacggttcttgcaggttgaacatcagcgcgctcttacctttcgctttcagttctttatccagcgccgggatctcttcccaggtttttggcgggttcggcagcagatctttgttataaatcagcgataacgcttcaacagcgatcgggtaagcaatcagcttgccgttgtaacgtacggcatcccaggtaaacggatacagcttgtcctggaacgctttgtccggggtgatttcagccaacaggccagattgagcgtagccaccaaagcggtcgtgtgcccagaagataatgtcagggccatcgccagttgccgcaacctgtgggaatttctcttccagtttatccggatgctcaacggtgactttaattccggtatctttctcgaatttcttaccgacttcagcgagaccgttatagcctttatcgccgttaatccagattaccagtttaccttcttcgattttcatatgacctcctaagcatcgatagatcctgtttcctgtgtgaaattgttatccgctcacaattccacacattatacgagccgatgattaattgtcaacagggggatggggagtaagctgatcctgtttcctgtgtgaaattgttatccgctcacaattccacacattatacgagccgatgattaattgtcaacagggggatggggagtaagctcatcgatggatcgatcctgtttcctgtgtgaaattgttatccgctcacaattccacacattatacgagccggaagcataaagtgtaaagcctggggtgcctaatgagtgagctaacttacattaattgcgttgcgctcactgcccgctttccagtcgggaaacctgtcgtgccaggacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccccaggctttacactttatgcttccggctcgtatggcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccattatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcag

SEQ ID NO: 4 - DNA, 29 bp, Artificial Sequence
  5′ modified restriction site
attccaattcgatcggggggggggggaaa

SEQ ID NO: 5 - DNA, 31 bp, Artificial Sequence
  3′ modified restriction site
attccaagtagtactttagaaaaactcatcg

SEQ ID NO: 6 - DNA, 83 bp, Artificial Sequence
  5′ modified multiple cloning site
atcgatcgacatatgggatccgagctcaagctttctagactcgaggaattcggtaccgtcgacatcgatgataagctgtcaaa

SEQ ID NO: 7 - DNA, 31 bp, Artificial Sequence
  3′ modified multiple cloning site
attccaagtagtactactcttcctttttcaa

SEQ ID NO: 8 - DNA, 32 bp, Artificial Sequence
  5′ PCR primer for pcWIN2 construct
caattatatagatctatcgatgcttaggaggt

SEQ ID NO: 9 - DNA, 36 bp, Artificial Sequence
  3′ PCR primer for pcWIN2 construct
ttgccttattctagatcattagtggtgatggtggtg

SEQ ID NO: 10 - DNA, 6659 bp, Artificial Sequence
  Custom DNA vector pMS39
tcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcctggcacgacaggtttcccgactggaaagcgggcagtgagcgcaacgcaattaatgtaagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatctatcgatgcttaggaggtcatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcggatctattgtggcgaccggcggcaccaccaccaccgcgaccccgaccggctccggcagcgtgacctcgaccagcaaaaccaccgcgaccgcgagcaaaaccagcaccagcacctcatcaacctcctgtaccaccccgaccgcggtggcggtgaccttcgatctgaccgcgaccaccacctacggcgaaaacatctacctggtgggctcgatctctcagctgggtgattgggaaaccagcgatggcattgcgctgagcgcggataaatacacctccagcgatccgctgtggtatgtgaccgtgaccctgccggcgggtgaatcgtttgaatacaaatttatccgcattgaaagcgatgattccgtggaatgggaaagcgatccgaaccgcgaatacaccgtgccgcaggcgtgcggcacctcgaccgcgaccgtgaccgatacctggcgcggatccgagctcaagctttctagactcgaggaattcggtaccgtcgacatcgatgataagctgtcaaacatgagcagatctgagcccgcctaatgagcgggcttttttttcagatctgcttgaagacgaaagggcctcgtgatacgcctatttttataggttaatgtcatgataataatggtttcttagacgatgcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttcatcgtcaggtggcacttttcggggaaatgtgcgcggaacccctatttgtttatttttctaaatacattcaaatatgtatccgctcatgagacaataaccctgataaatgcttcaataatattgaaaaaggaagagtagtactttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaatagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacagtcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgccatacgagccggaagcataaagtgtaaagcctggggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccag

SEQ ID NO: 11 - DNA, 6647 bp, Artificial Sequence
  Custom DNA vector pMXS39
tcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcctggcacgacaggtttcccgactggaaagcgggcagtgagcgcaacgcaattaatgtaagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatctatcgatgcttaggaggtcatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcggatccgagctcaagctttctagactcgagattgtggcgaccggcggcaccaccaccaccgcgaccccgaccggctccggcagcgtgacctcgaccagcaaaaccaccgcgaccgcgagcaaaaccagcaccagcacctcatcaacctcctgtaccaccccgaccgcggtggcggtgaccttcgatctgaccgcgaccaccacctacggcgaaaacatctacctggtgggctcgatctctcagctgggtgattgggaaaccagcgatggcattgcgctgagcgcggataaatacacctccagcgatccgctgtggtatgtgaccgtgaccctgccggcgggtgaatcgtttgaatacaaatttatccgcattgaaagcgatgattccgtggaatgggaaagcgatccgaaccgcgaatacaccgtgccgcaggcgtgcggcacctcgaccgcgaccgtgaccgatacctggcgctaatgagtcgacatcgatgataagctgtcaaacatgagcagatctgagcccgcctaatgagcgggcttttttttcagatctgcttgaagacgaaagggcctcgtgatacgcctatttttataggttaatgtcatgataataatggtttcttagacgatgcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttcatcgtcaggtggcacttttcggggaaatgtgcgcggaacccctatttgtttatttttctaaatacattcaaatatgtatccgctcatgagacaataaccctgataaatgcttcaataatattgaaaaaggaagagtagtactttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaatagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacagtcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgccatacgagccggaagcataaagtgtaaagcctggggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttt

SEQ ID NO: 12 - DNA, 35 bp, Artificial Sequence
  5′ PCR primer for pCWin2-MBP-MCS-SBD (pMXS39) expression vector
tgtatcctcgagattgtggcgaccggcggcaccac

SEQ ID NO: 13 - DNA, 38 bp, Artificial Sequence
  3′ PCR primer for pCWin2-MBP-MCS-SBD (pMXS39) expression vector
aagcttgtcgactcattagcgccaggtatcggtcacgg